(19) United States
(12) Patent Application Publication
(10) Pub. No.: US 2020/0188540 A1
(43) Pub. Date: Jun. 18, 2020

(54) CONFORMATIONALLY STRAINED TRANS-CYCLOALKENES FOR RADIOLABELING

(71) Applicants: UNIVERSITY OF DELAWARE, Newark, DE (US); THE UNIVERSITY OF NORTH CAROLINA AT CHAPEL HILL, Chapel Hill, NC (US); UNITED KINGDOM RESEARCH AND INNOVATION, Swindon (GB)

(72) Inventors: Joseph FOX, Landenberg, PA (US); Zibo LI, Chapel Hill, NC (US); Yu LIU, Kansas City, MO (US); Michael Thompson TAYLOR, Ely (GB); Dennis SVATUNEK, Vienna (AT); Katarina ROHLFING, Wilmington, DE (US); Mengzhe WANG, Chapel Hill, NC (US); Zhanhong WU, Chapel Hill, NC (US); Raghu VANNAM, Streetsboro, OH (US); Jason W. CHIN, Cambridge, Cambridgeshire (GB); Stephen WALLACE, Cambridge, Cambridgeshire (GB)

(73) Assignees: University of Delaware, Newark, DE (US); The University of North Carolina at Chapel Hill, Chapel Hill, NC (US); United Kingdom Research and Innovation, Swindon (GB)

(21) Appl. No.: 16/062,756

(22) PCT Filed: Dec. 14, 2016
(86) PCT No.: PCT/US2016/066504

§ 371 Date: Jun. 15, 2018

Related U.S. Application Data
(60) Provisional application No. 62/267,441, filed on Dec. 15, 2015.

(51) Int. Cl. A61K 51/08; C07F 1/08; C07F 7/08; C07F 5/00; C07D 313/18; C07D 405/12; C07D 317/44; C07D 403/12; C07D 225/00

(52) U.S. Cl. CPC A61K 51/082; C07F 1/08; C07F 7/0807; C07F 7/0816; C07D 225/00; C07D 313/18; C07D 405/12; C07D 317/44; C07D 403/12; C07F 5/00

(57) ABSTRACT

Conformationally strained irans-cycloalkenes and derivatives thereof suitable for radiolabeling in a subject in need thereof.

This application claims priority benefit of U.S. Application No. 62/267,441, filed 15 Dec. 2015, the entire contents of which are incorporated herein by reference for all purposes.

### BACKGROUND OF THE INVENTION

Positron emission tomography (PET) is a non-invasive imaging modality with the capacity to track radiolabeled biomolecules in vivo. This imaging technique employs radionuclides that emit positrons that collide with electrons and result in two detectable γ-rays. Of the common radionuclides that are utilized in PET, ¹⁸F is the most broadly utilized due to the high positron efficiency, high specific radioactivity and clinically attractive half-life (˜110 min). These properties can minimize the toxic effects and radiation exposure to the patient. However, the short half-life of ¹⁸F, the modest nucleophilicity of fluoride, and the low concentrations that are intrinsic to both biology and radiochemistry render it challenging to incorporate ¹⁸F in complex biomolecules. Accordingly, there is a high demand for compounds and methods that efficiently introduce ¹⁸F into biological macromolecules.

### SUMMARY OF THE INVENTION

The invention provides conformationally strained trans-cycloalkenes and derivatives thereof suitable for use in radiolabeling in a subject in need thereof.

### BRIEF DESCRIPTION OF THE DRAWINGS

FIG. 1 shows the quantitative biodistribution derived from small-animal PET images, showing localization of ¹⁸F-15 in human U87MG tumor-bearing mice (n=5), performed by static microPET scans.

FIG. 2A shows images depicting tumor uptake of a radiolabeled imaging agent according to the invention.

FIG. 2B shows quantitative activity distribution in blood samples obtained in the animals shown in FIG. 2A, suggesting interaction between imaging probes and serum proteins.

### DETAILED DESCRIPTION OF THE INVENTION

As used herein, descriptions and structural representations of fluorine-containing compounds are to be understood to apply both to compounds in which the fluorine is ¹⁸F and those in which it is ¹⁹F, unless made otherwise clear by the context or by explicit notation identifying the isotope.

The inventors now disclose a variety of strained trans-cycloalkenes and sila-trans-cycloalkenes, and derivatives of these compounds useful as radiotracers, for example in PET imaging. Among other uses, these compounds can adduct to tetrazines, thereby providing means of providing orthogonal coupling reactions for use in vivo. Although all of the halogenated compounds explicitly described herein are fluorinated compounds, the skilled person will be able to prepare analogs using any other halogen, and all of these compounds and uses thereof are to be considered as being according to the invention. Thus, for example, any isotope of Cl, Br, I, or At can be used. For instance, ¹²⁴I and ¹³¹I may be used.

For example, the inventors now disclose the ¹⁸F version of compound 9, referred to herein as ¹⁸F-9, a new radiotracer of extremely high reactivity as a dienophile. Although the syn diastereomer is shown herein for compound ¹⁸F-9, structural diagrams and references to ¹⁸F-9 will be understood to apply to both the syn and anti diastereomers unless the context makes otherwise clear. The same applies to all other compounds discussed herein.

Although compound ¹⁸F-9 is shown as comprising three ethylene oxide repeat units in the chain, the number may instead be 1 or 2, or any integer. Typically, the number of ethylene oxide repeat units will be at least 3, or at least 5, 10, or 20. The number will typically be at most 100, or at most 50, 40, or 30. Or, the number n may correspond to the number of repeat ethylene oxide units in any polyethylene oxide or polyethylene glycol polymer. That is, the group may be a polyethylene oxide or polyethylene glycol linking group. These same numbers and ranges of ethylene oxide units also apply as optional modifications to any compound comprising ethylene oxide units disclosed herein.

Compound ¹⁸F-9 rapidly combines with tetrazines and can be used to rapidly assemble probes for PET imaging. The kinetics in Diels-Alder reactions of the two diastereomeric compounds 5 and 4 were evaluated, and the more reactive syn-sTCO diastereomer was utilized for further study in PET probe construction. The tetrazine ligation with ¹⁸F-9 was used to synthesize a radiolabeled RGD peptide and in a mouse tumor model was demonstrated to have a high level of tumor uptake relative to that in liver, kidney, and muscles. At 4 h post injection, the tumor was the most prominent image in the PET scan, with tumor uptake that was 1.6-2.4 fold higher than for other major organs. The anti-diastereomer (“anti-sTCO”) 4 was prepared as described previously (Taylor, M. T.; Blackman, M. L.; Dmitrenko, O.; Fox, J. M. J. Am. Chem. Soc. 2011, 133, 9646-9649), and the syn-diastereomer (rel-1R,8S,9S,4E)-bicyclo[6.1.0]non-4-ene-9-ylmethanol 5 (“syn-sTCO”) was prepared as shown in Scheme 1(A). Thus, photoisomerization of 7 using the inventors' previously described flow reactor gave syn-sTCO 5 in 81% yield. The inventors' initial efforts to activate syn-sTCO 5 through reaction with NsCl or TsCl were unsuccessful, and led only to skeletal rearrangement products. After experimentation, the inventors developed a synthesis that directly provided a tosylate product through alkylation of the alcohol 5 with a bis-tosylate that contained a mini-PEG linker. Thus, combination of this alcohol with KH and triethylene glycol ditosylate gave the sTCO tosylate 8 in 28% yield. To create the HPLC standard, the treatment of 8 with TBAF in anhydrous THF gave the ¹⁹F-labeled derivative 9 in 76% yield. A diphenyl-s-tetrazine conjugate of a cyclic RGD was synthesized as shown Scheme 1(B). The nitrophenylcarbonate 10 was sequentially coupled with a “mini-PEG” amino acid to give 11. Subsequent coupling with NHS and conjugation with the cyclic peptide RGDyK gave 12 in high yield.

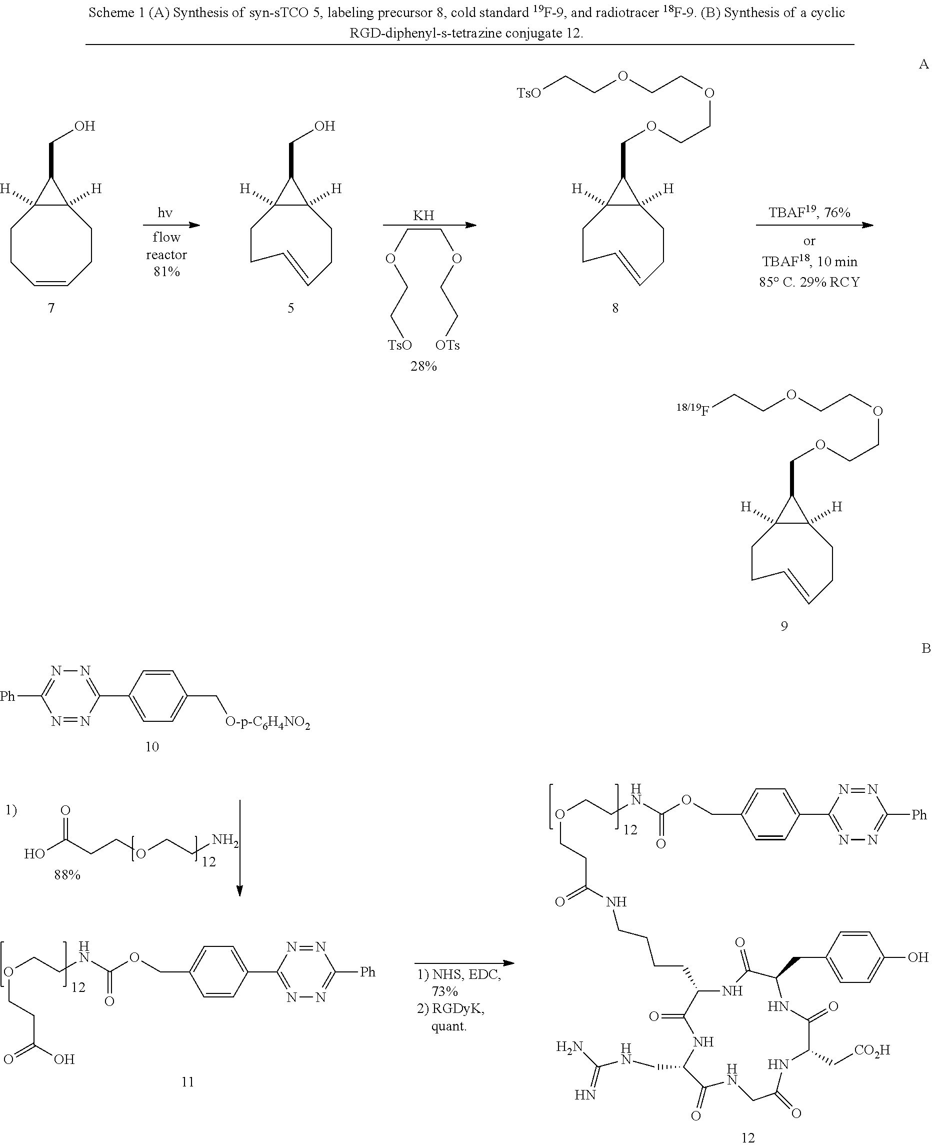

Stopped flow kinetic analysis was used to measure the rate of the Diels-Alder reaction between tetrazine derivative 11 and anti- and syn-diastereomers of sTCO (4 and 5, respectively). In an earlier study with 13, a mini-PEG derivative of the sTCO anti-diastereomer, it was found that the water soluble diphenyl-s-tetrazine analog 14 and the di-2-pyridyl-s-tetrazine analog 16 react with rate constants of 2.86×10⁵ M⁻¹s⁻¹ and 3.3×10⁶ M⁻¹s⁻¹. The latter is the fastest rate constant that has been described for a bioorthogonal reaction. For the present study, the inventors used stopped flow analysis to compare the relative rate of the syn- and anti-diastereomers 5 and 4 with tetrazine 11 in mixed organic/aqueous media (55:45 MeOH:water at 25° C.). As expected, the rates in MeOH:water were ˜9 fold slower than the measurements made in purely aqueous media, but still extremely rapid. The rate constant for the syn-diastereomer 5 with tetrazine 11 was k₂ 3.7×10⁴ (+/−0.1×10³) M⁻¹s⁻¹, and the anti-diastereomer 4 reacted with a rate constant k₂ 3.3×10⁴ (+/−0.1×10³) M⁻¹s⁻¹. Because the syn-diastereomer was more reactive it was chosen for further development

Radiochemistry ¹⁸F-labeled sTCO (¹⁸F-9) was produced using the protocol described in Scheme 1. By treating tosylate precursor 8 (182 mM) with ¹⁸F-TBAF in acetonitrile at 85° C. for 10 min, the inventors were able to obtain the radiolabeled ¹⁸F-9 in 29.3+/−5.1% isolated radiochemical yield with 99% radiochemical purity after HPLC purification. (Other ¹⁸F sources also worked, such as ¹⁸F-KF/K222.)

Here, the reaction concentration was determined to be important, as running the reaction at 91 mM gave ¹⁸F-9 in only 9.3+/−2.4% isolated yield. The specific activity was determined to be 2.1+/−0.8 Ci/μmol. The product identity was confirmed by co-injection with an independently synthesized ¹⁹F-9 standard. Prior to performing reactions with targeting molecules, the inventors first tested the in vitro stability of ¹⁸F-9. After incubation in 1× PBS, the radiopurity remained at 97.5% and 97.3% at 1 hour and 2 hour time points, respectively. This result demonstrated that ¹⁸F-9 is sufficiently stable to construct PET probes in aqueous solution. It was also observed that ¹⁸F-9 was stable in fetal bovine serum for 1 hour with retention of 74% radiochemical purity.

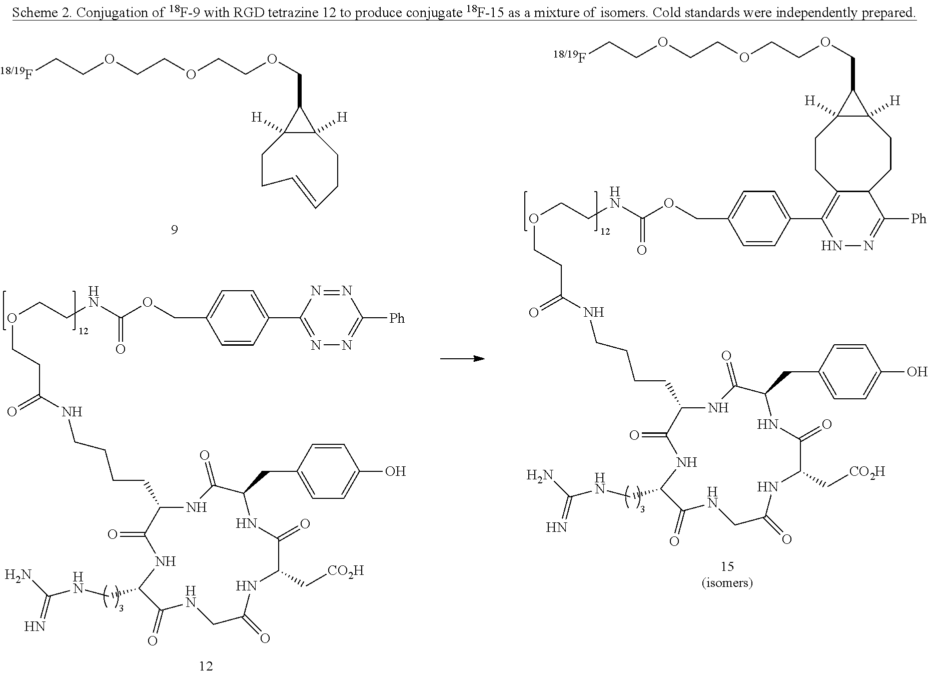

As depicted in Scheme 2, the conjugation of ¹⁸F-9 with RGD-tetrazine 12 (700 μM) produced conjugate ¹⁸F-15 as a mixture of isomers. The starting material ¹⁸F-9 was completely consumed upon initial assay (<5 minutes). Reducing the concentration of 12 to 33 μM lead to an inversion in stoichiometry, and the complete consumption of 12 and the observation of unreacted ¹⁸F-9. This ability to achieve complete labeling when the ¹⁸F-labeled substrate is used in excess speaks to the high efficiency and rate of bioorthogonal reaction using ¹⁸F-9.

Under ambient reaction conditions, a 91% radiochemical yield of ¹⁸F-15 was obtained with 99% purity after HPLC purification. The specific activity was determined to be 0.91+/−0.20 Ci/μmol. An analog reaction with ¹⁹F-9 produced the isomeric “cold” Diels-Alder conjugates ¹⁹F-15. LC-MS analysis confirmed that the both of the major peaks from the conjugation had mass spectra matching the theoretical for ¹⁹F-15. More rapidly eluting minor peaks also had correct mass data, and likely correspond to the aminal (hydrated) forms of the product. The slowest eluting peak from the radio-HPLC trace of ¹⁸F-15 was collected and the in vitro stability was studied. It was observed that the adduct was stable in PBS buffer for 2 hours with retention of 98.5% radiochemical purity. Conjugate ¹⁸F-15 was also found to be stable in fetal bovine serum with 96.7% and 94.5% purity at 2 and 4 hours post incubation respectively.

Due to the low concentration and short time scale that is intrinsic to ¹⁸F labeling of proteins, the fast kinetics and bioorthogonality of the tetrazine-TCO ligation provide a clear benefit over conventional radiolabeling methods. In previous work, the inventors found that reactive tetrazines were required in order to obtain rapid reactivity at micromolar concentrations, but the resulting Diels-Alder conjugates had only moderate stability in vivo. The superior reactivity of ¹⁸F-9 allows rapid kinetics (>10⁴ M⁻¹s⁻¹) to be realized with more stable diphenyl-s-tetrazines, giving rise to Diels-Alder conjugates having improved in vivo stability. Moreover, the system described here leads to conjugates with improved blood circulation and higher levels of tumor uptake than observed using those described previously.

Small Animal PET Imaging

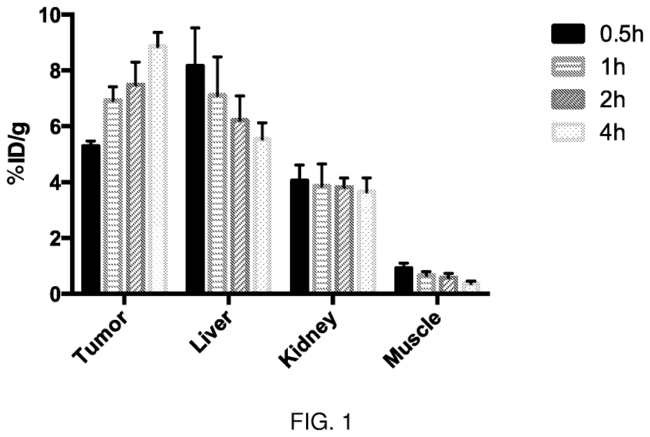

The localization of ¹⁸F-15 in human U87MG tumor-bearing mice (n=5) was performed by static microPET scans at multiple time-point post tail vein injection. Selected decay-corrected coronal images at different time points were obtained after injection of 3.7 MBq (100 μCi) of ¹⁸F-15. High and persistent tumor accumulation was observed with good tumor to background contrast as early as 30 min post injection. The quantitative biodistribution derived from small-animal PET images are shown in FIG. 1, showing tumor and major organ radioactivity accumulation quantification from a static scan at 0.5, 1, 2, and 4 h post injection of ¹⁸F-15 into U87MG tumor model. Data are expressed as average +/−SD.

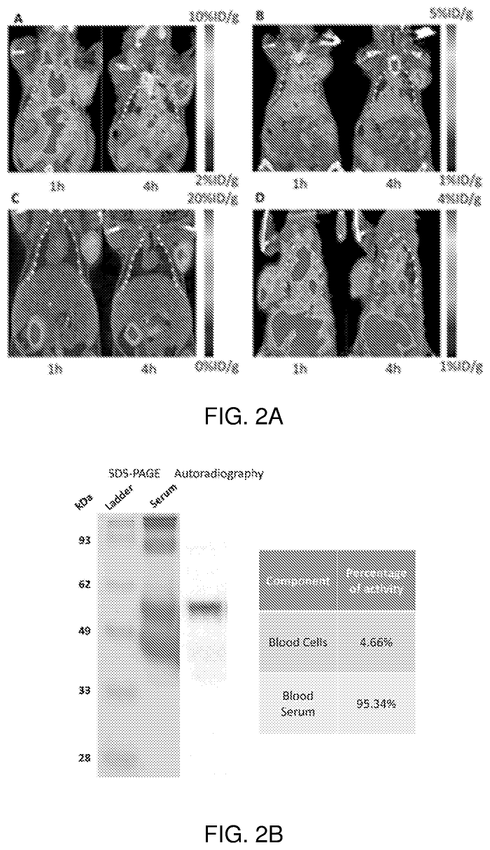

The inclusion of mini-PEG spacers resulted in a biodistribution profile that was significantly improved relative to previously constructed TCO/tetrazine-based probes that lack a PEG spacer, and the blood circulation of this new construct was improved significantly compared with previously described constructs. The tumor uptake was 5.3+/−0.2, 6.9+/−0.5, 7.5+/−0.8 and 8.9+/−0.5% ID/g at 0.5, 1.0, 2.0, and 4.0 h post injection, respectively. At 4.0 h post injection, the tumor became the brightest spot in PET scan, with a tumor-to-liver and tumor-to-kidney ratio of 1.6 and 2.4, respectively. Given the improved blood circulation and high levels of tumor uptake for this small peptide-based probe, the inventors anticipate that ¹⁸F-9 based probes should find broad utility for the labeling a variety of biomolecules, including peptides, proteins, antibodies, oligonucleotides, and nanoparticles. Indeed, additional PET agents are prepared based on the ¹⁸F-9 and diphenyl-tetrazine system. See Scheme 3. As seen in FIG. 2A and FIG. 2B, the blood circulation of traditional fast clearing peptides was significantly increased, leading to increased or persistent tumor uptake. Further investigation suggests the enhanced blood circulation is caused by the binding/interaction with serum proteins. The system should also be applicable to other molecules and biologics for the development of long-acting therapeutic drugs.

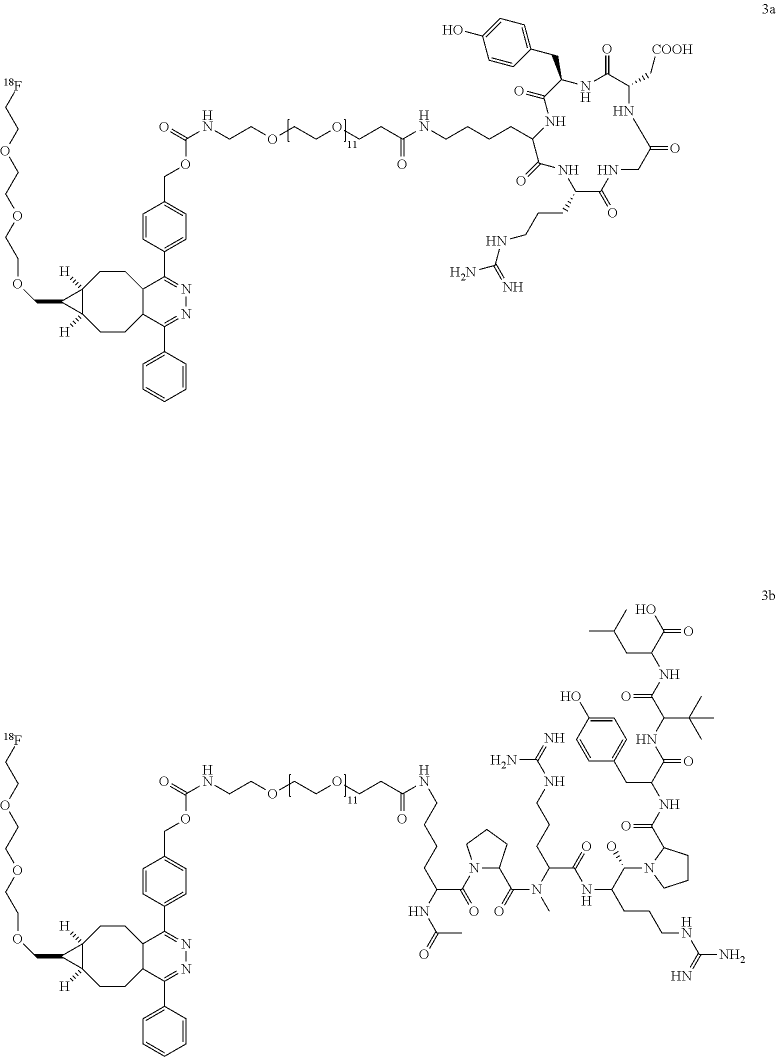

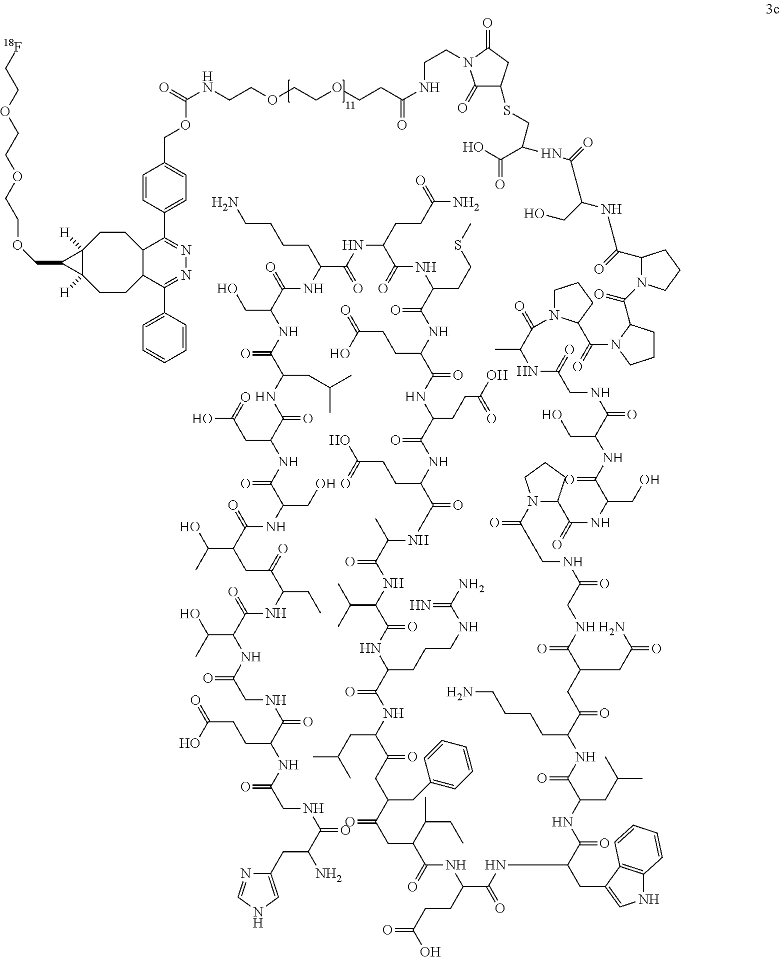

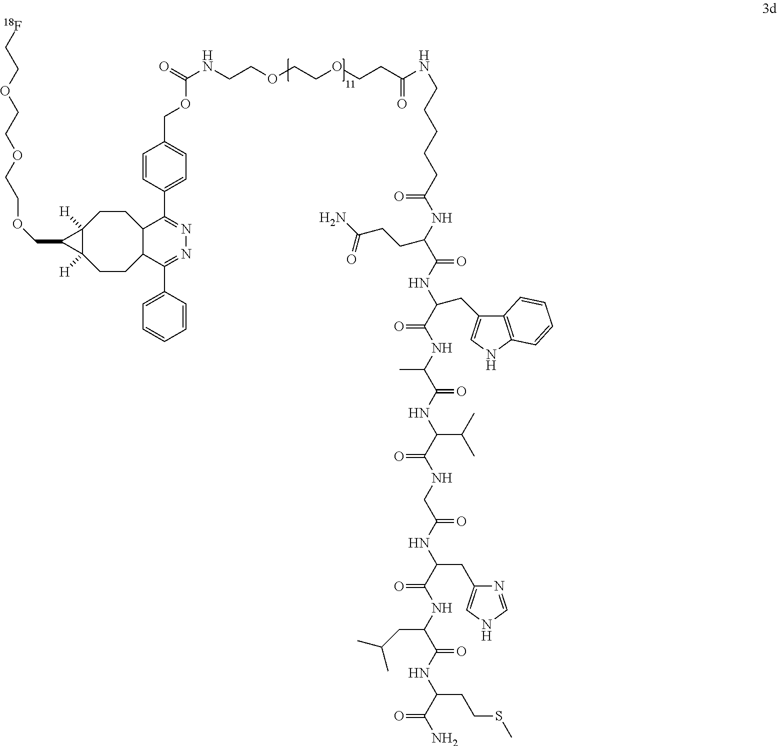

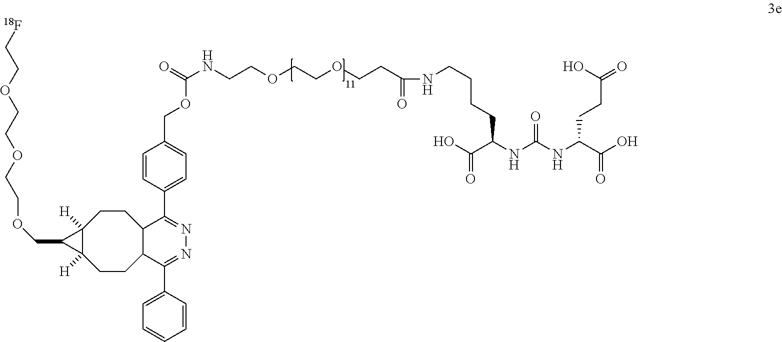

The specificity of ¹⁸F-15 was confirmed by a blocking experiment in which the radiotracer was co-injected with an excess amount of cRGDyK. The RGD peptide is a well-established targeting molecule. In the presence of non-radio labeled cRGDyK (200 μg), the tracer uptake in tumor dropped to 4.8+/−0.3% at 1 h post injection. As expected the cRGDyK peptide, which should be readily cleared than a PEGylated peptide, did not completely block the signal due to ¹⁹F-15. However, the signal in the presence of blocking cRGDyK was significantly (P<0.05) lower than that observed without a blocking agent.

The inventors also performed microPET imaging with a normal (non-tumor bearing) nude mouse that had been injected with ¹⁸F-9. The imaging data indicated that the compound was rapidly cleared by the gallbladder, kidney and liver within 2 hours. The inventors also analyzed the clearance of the compound obtained by combining ¹⁸F-9 with 11. This Diels-Alder conjugate—and analog of ¹⁸F-15 that lacks the RGD moiety—still remained in the blood circulatory system after 4 hours. The blood uptake was 2.4% ID/g at 4 h post injection. The inventors have previously reported ¹⁸F-labeled RGD probes derived from trans-cyclooctene 1.

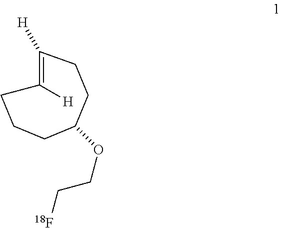

These probes, which lack PEGylation, are cleared much more rapidly. Without wishing to be bound by any particular explanation, the inventors believe these results suggest that the entire PEGylated Diels-Alder moiety plays a role in enhancing the circulation time of the probe. The inventors believe that the rapid clearance of ¹⁹F-9 and the long circulation lifetime of its PEGylated Diels-Alder conjugates may prove advantageous for applications in pretargeted imaging.

Other Conformationally Strained Trans-Cycloalkenes

The invention also provides conformationally strained trans-cyclooctene structures that possess cis-ring fusions, with general structures represented as 22 and 23, including but not limited to the general structures 24-26. The invention also provides derivatives of 22-26 where a radiolabel is attached, either directly to the structure, or through a tether. In structures of type 22-26, the cis-ring junction causes the 8-membered ring to adopt a more reactive ‘half chair’ conformation. This differs from ordinary trans-cyclooctenes, which adopt a less reactive ‘crown’ conformation.

The invention also provides structures of the general type 27 and 28, where additional olefinic strain is introduced through the inclusion of heteroatoms in the backbone of the trans-cyclooctene. Here, the shorter bonds to heteroatoms introduce additional angle strain to the olefin. The invention also provides derivatives of these compounds where a radiolabel is attached, either directly to the structure, or through a tether. The invention also provides structures of type 29, where olefinic strain is increased through a decrease in ring size to a sila-trans-cycloheptene. The invention also provides derivatives of these compounds where a radiolabel is attached, either directly to the structure, or through a tether. In structures 24-29, R is any conjugatable functional group, including OH, CH₂OH, or CO₂H; where R′ is Me or a conjugatable functional group, including OH, CH₂OH, or CO₂H, and where R″ is H or a conjugatable functional group, including OH, CH₂OH, or CO₂H.

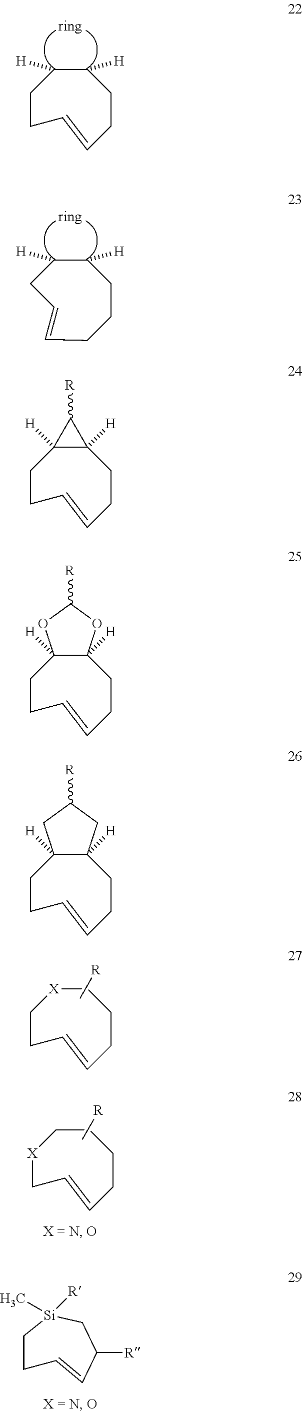

The inventors have also prepared the following complexes.

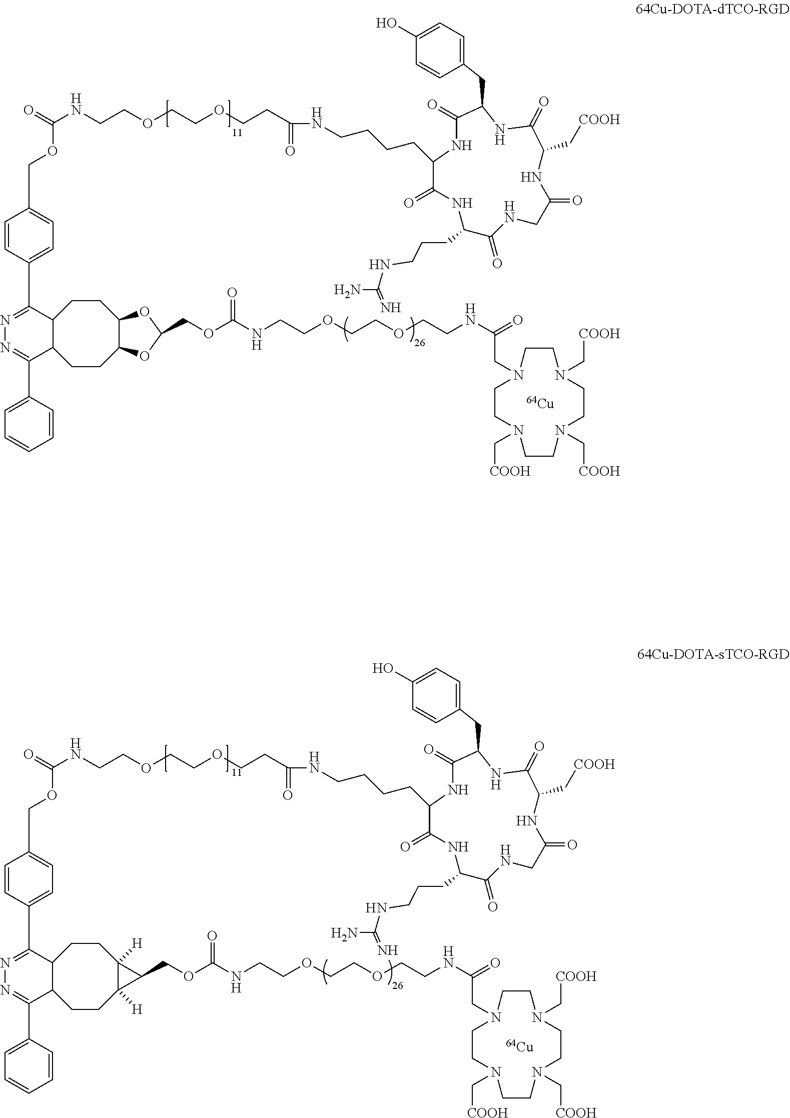

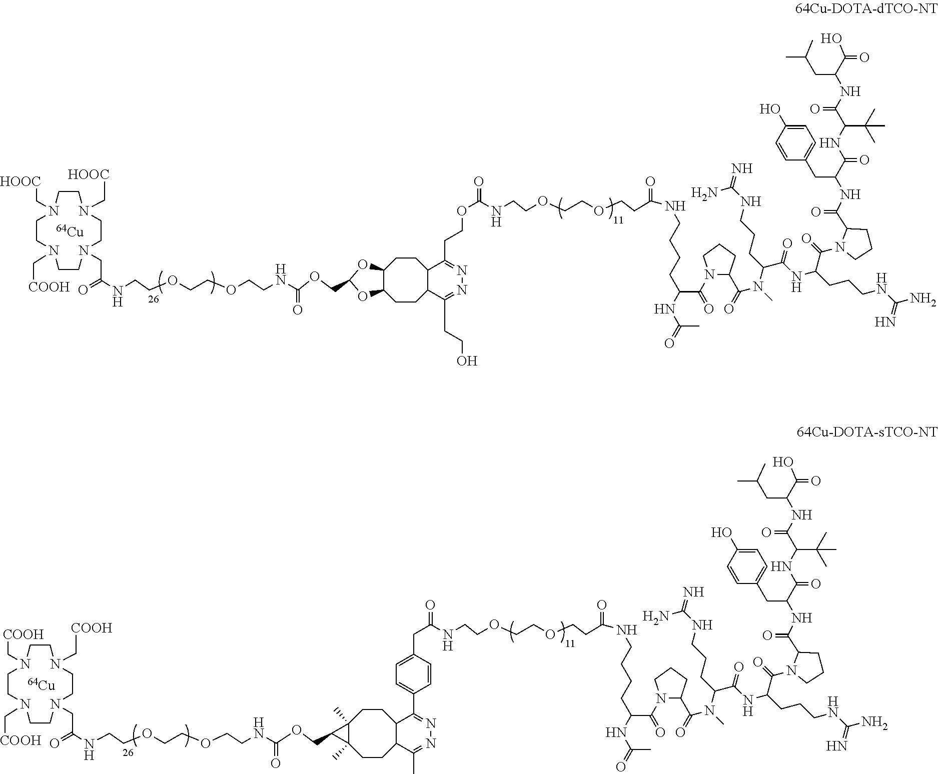

Additional compounds according to the invention are disclosed in the Examples. The invention also provides the following compounds, all of which can be made by the skilled person by appropriately modifying, where needed, the methods disclosed herein.

In the following compounds, the number n as applied to ethylene oxide repeat units can be 1, 2, 3, or any integer. Typically, the number of ethylene oxide repeat units will be at least 3, or at least 5, 10, or 20. The number will typically be at most 100, or at most 50, 40, or 30. Or, the number n may correspond to the number of repeat ethylene oxide units in any polyethylene oxide or polyethylene glycol polymer. That is, the group may be a polyethylene oxide or polyethylene glycol linking group. The notation “LG” represents halogen or sulfonate. The notation M in the complexes shown below is any radioactive or non-radioactive isotope of any metal. Examples include ⁶⁴Cu, ⁶⁷Cu, ⁸⁶Y, ⁹⁰Y, ¹⁷⁷Lu, Gd, and Ln. R and R′ are each individually chosen from H and methyl.

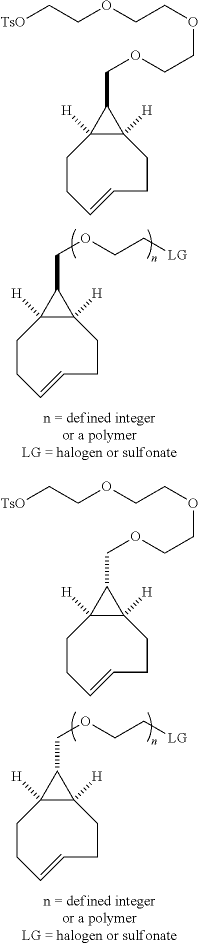

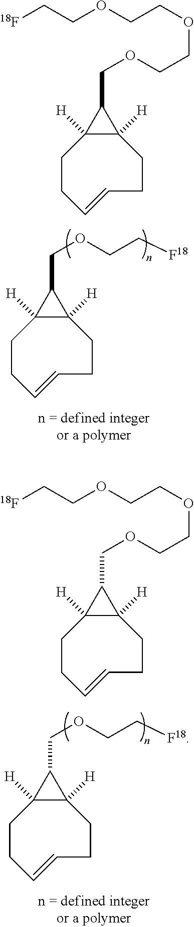

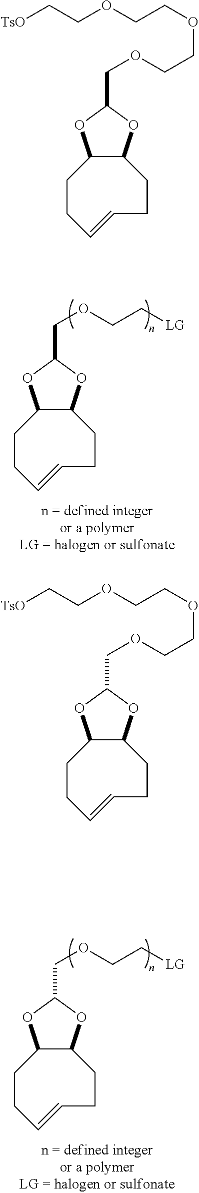

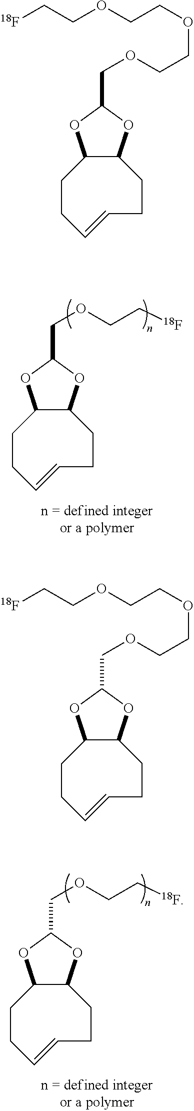

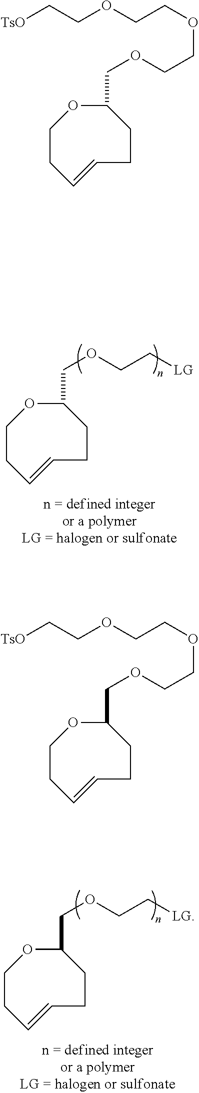

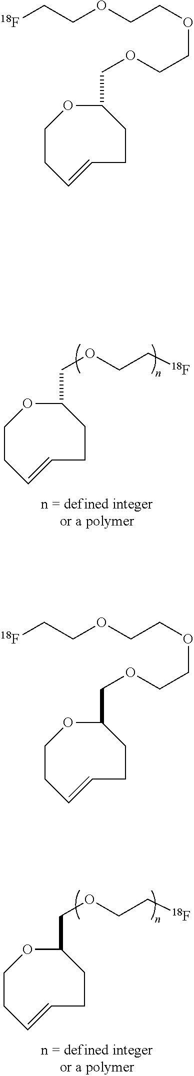

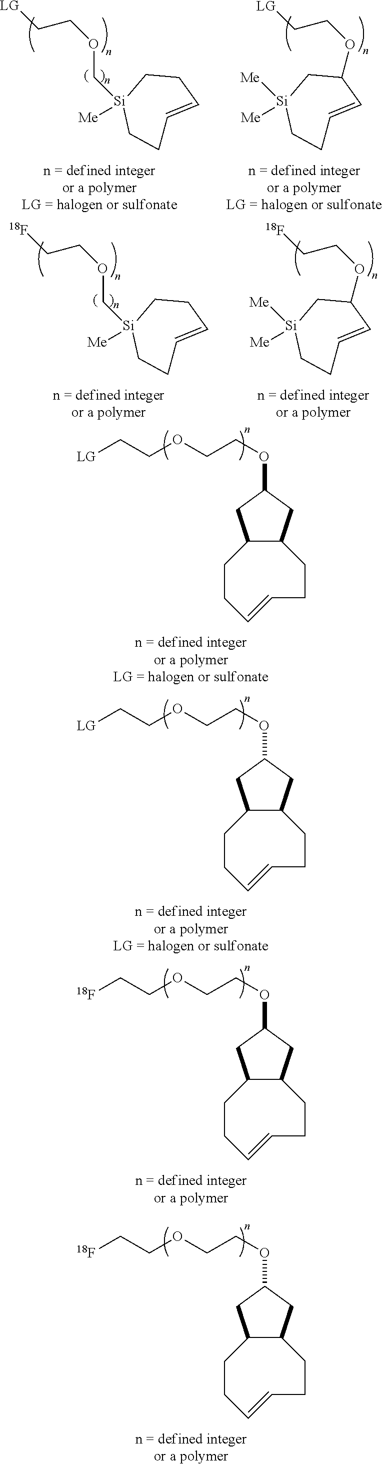

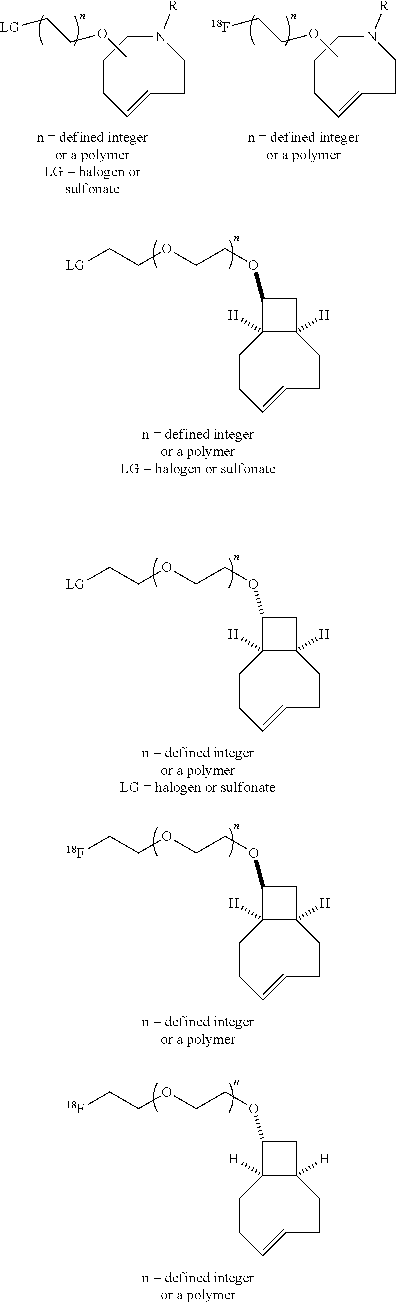

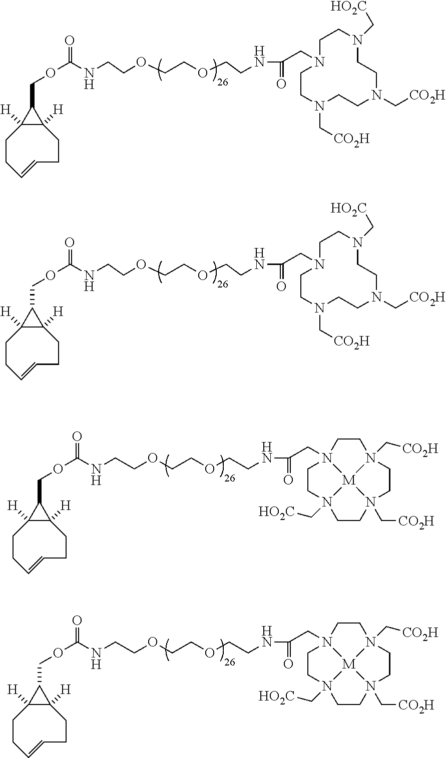

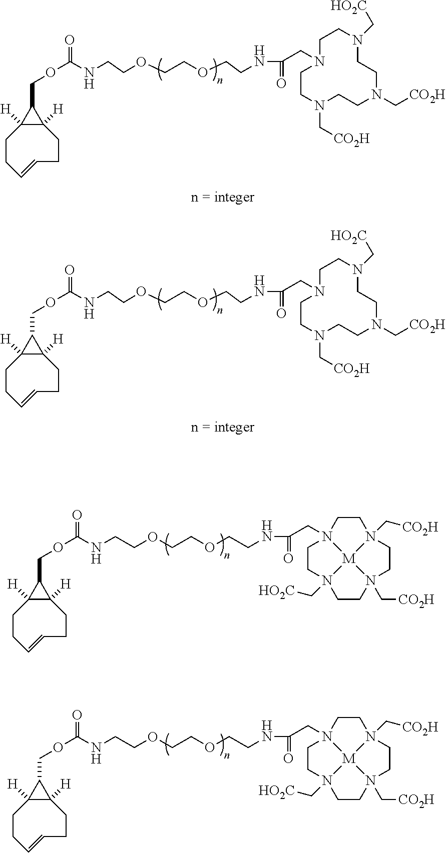

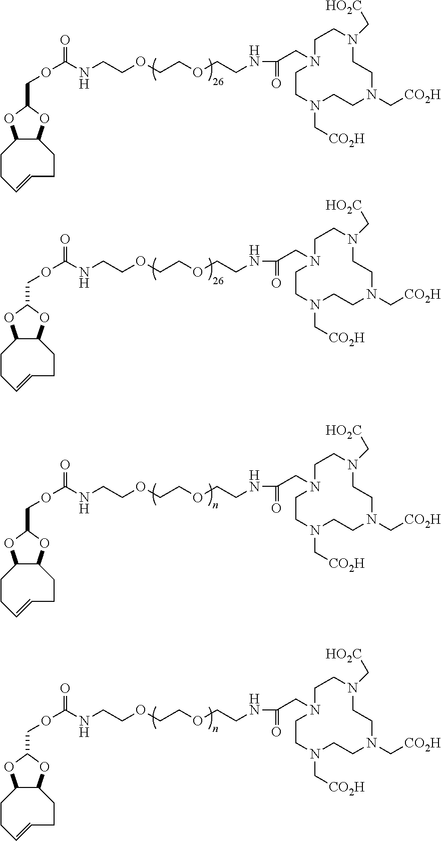

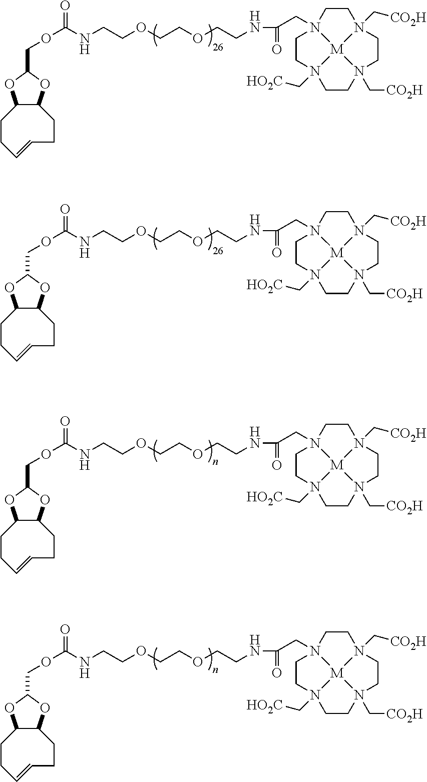

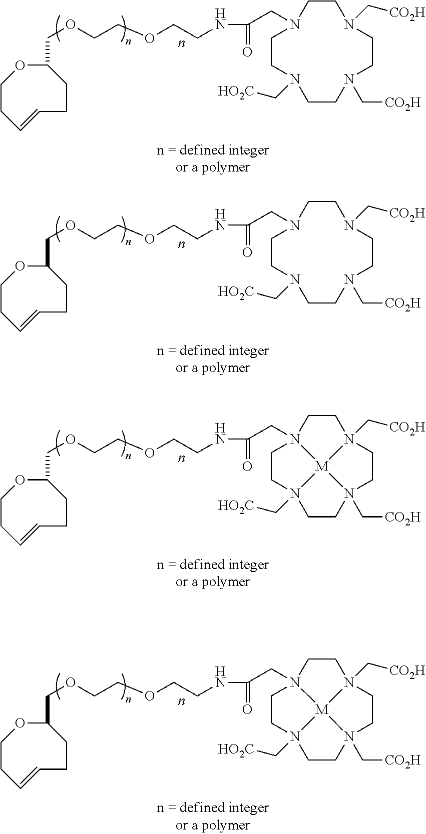

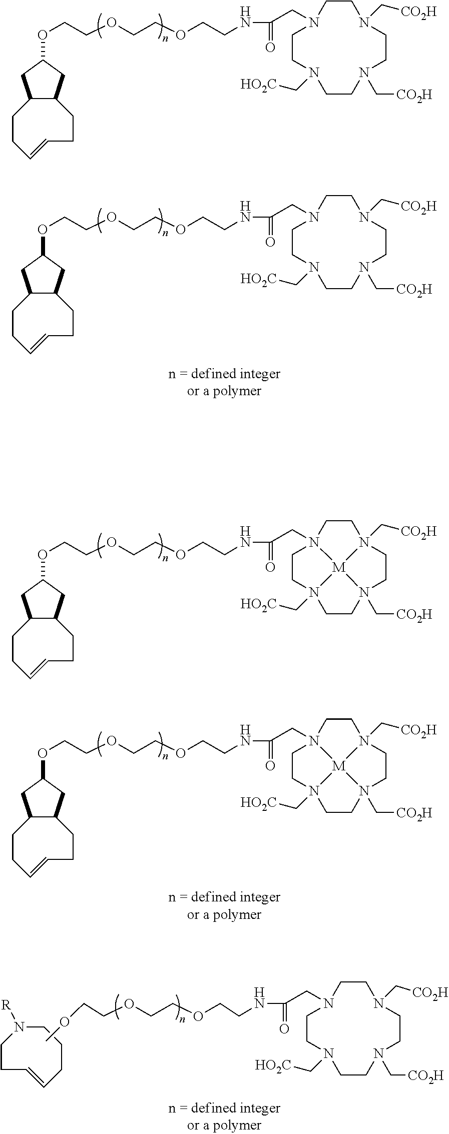

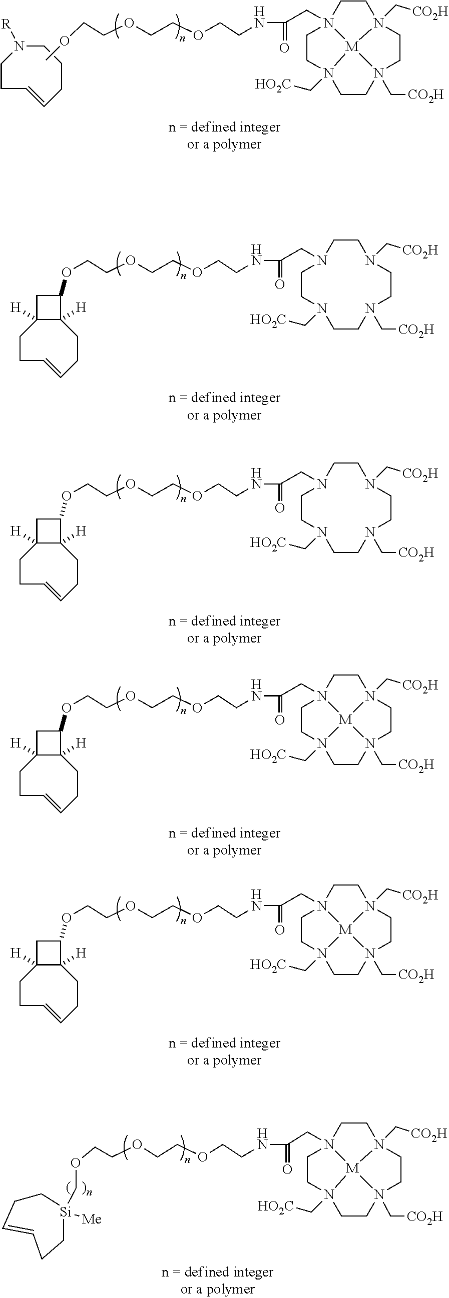

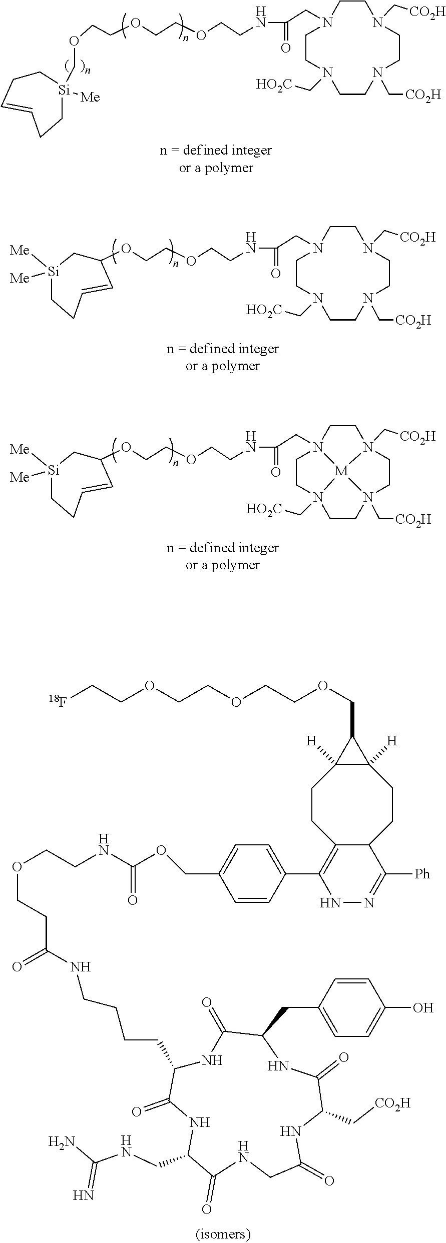

The term “isomers” in the last compound immediately above refers to isomers of the dihydrotetrazine moiety.

Any of the ¹⁸F compounds disclosed herein may be injected into a subject in need of PET imaging.

### EXAMPLES

Materials and Methods

All commercially available analytical grade chemical reagents were purchased from Aldrich (St. Louis, Mo.) and used without further purification. Analytical reversed-phase HPLC using a Gemini 5μ C18 column (250×4.6 mm) was performed on a SPD-M30A photodiode array detector (Shimadzu) and model 105S single-channel radiation detector (Carroll & Ramsey Associates). Radio HPLC analyses were carried out at 1 mL/min with water/acetonitrile eluent mixtures. For other HPLC analyses, the solvents were modified with 0.1% trifluoroacetic acid.

Stopped-Flow Kinetic Analysis

The second order rate constant was measured under pseudo-first order conditions using an excess of the appropriate sTCO diastereomer (4 or 5), and by following the exponential decay of absorbance due to the tetrazine chromophore of 11 at 298 nm using an SX 18MV-R stopped-flow spectrophotometer (Applied Photophysics Ltd.). For each run, equal volumes of 45:55 water:methanol solutions of sTCO and PEGylated tetrazine 11 were mixed in the stopped flow device. Reactions were carried out with tetrazine 11 at 0.05 mM and final concentrations of 0.245, 0.49, 0.98 and 1.47 mM for the syn-diastereomer 5. Similarly, reactions were carried out with tetrazine 11 at 0.05 mM and final concentrations of 0.25, 0.50, 1.00 and 1.50 mM for the anti-diastereomer 4. A total of 400 data points were recorded over a period of 1 second, and each sample was performed in sextuplicate at 298 K. The k_{obs} was determined by nonlinear regression analysis of the data points using Prism software (v. 6.00, GraphPad Software Inc.). The results are shown in Table 1.

Radiochemistry

The radiolabeling reactions were carried out using the following protocol unless specified. The sTCO-tosylate 8 (9.1 μmol) was dissolved in MeCN (30 μL) and then allowed to react with ¹⁸F-TBAF (200 mCi) at 85° C. for 10 min. The reaction was quenched by adding water (500 μL). The mixture was then passed through a Sep-Pak cartridge (Sep-Pak Plus light alumina) followed by HPLC purification. After HPLC purification, the fraction containing the desired product was diluted with 10 mL of water, trapped on C18 Sep-Pak, washed with 10 mL water, and eluted off with 0.5 mL EtOH. A portion of the solution containing ¹⁸F-9 was reserved for the in vitro stability test. Then a fraction of the solution (10 mCi, estimated to be 4.8 nmol) was mixed with a DMSO solution of tetrazine-RGD conjugate 12 (ranging from 0.07 μmol to 3.3 nmol). After shaking for 10 seconds at room temperature, a portion of the reaction mixture (3 mCi) was loaded onto HPLC for further analysis. The HPLC eluent containing ¹⁸F-15 was collected and organic solvent was removed using rotary evaporator. After carefully adjusting the pH to 7.5, ¹⁸F-15 was reconstituted in 1× PBS for the stability test and small animal studies.

In Vitro Stability

¹⁸F-9 and ¹⁸F-15 were each incubated in 1× PBS buffer at 37° C. An aliquot of the solution (˜25 μCi) was taken out and loaded on HPLC at 1 h and 2 h time points for analysis. ¹⁸F-9 was also incubated in FBS at 37° C. and after 1 h, an aliquot of the solution (˜25 μCi) was taken out and added to an equal volume of TFA. Similarly, ¹⁸F-15 was also incubated in FBS at 37° C., and at 2 and 4 h time points, aliquots of the solution (˜25 μCi) were taken and added to an equal volume of TFA. For each sample, the mixture was centrifuged at 14000 rpm for 5 min. The supernatant was then diluted with 1 mL water and loaded on C18 Sep-Pak. After washing with 1 mL water, the cartridge was eluted with 0.5 mL acetonitrile. The water fraction and acetonitrile fraction were combined and loaded on HPLC for analysis

Small Animal PET Imaging

Animal procedures were performed according to a protocol approved by the UNC Institutional Animal Care and Use Committee. PET scans and image analysis were performed using a small animal PET scanner. Human U87MG tumor-bearing mice were anesthetized using 2% isoflurane and injected with 3.7 MBq (100 μCi) of ¹⁸F-15 via the tail vein. At 0.5, 1.0, 2.0, and 4.0 h post injection, static emission scans were acquired for 10 min. Normal nude mice were injected with 3.7 MBq (100 μCi) of the Diels-Alder conjugate obtained by combining ¹⁸F-9 and 11), or in a separate experiment by injecting only ¹⁸F-9 using the same protocol. Raw PET images were reconstructed using 2D ordered subset expectation maximization (OSEM) algorithm. No background correction was performed. Regions of interest (ROI) were manually drawn over the tumor and other organs on the decay corrected coronal images. Based on the assumption that the tissue density is 1 g/mL, the ROIs were converted to % ID/g by dividing dose per gram at ROI by injected dose.

Statistical Analysis

Quantitative data were expressed as mean±SD. Means were compared using one-way ANOVA and Student's t test. P values<0.05 were considered statistically significant.

Synthetic Procedures

General Considerations: All reactions were carried out in glassware that was flame-dried under vacuum and cooled under nitrogen. All commercially available reagents and solvents were used as received. (rel-1R,8S,9S,4Z)-Bicyclo[6.1.0]non-4-ene-9-ylmethanol and 4-nitrophenyl 4-(6-phenyl-1,2,4,5-tetrazin-3-yl)benzyl carbonate were prepared following known procedures. Reactions were monitored by thin layer chromatography (TLC) performed on SiliCycle silica gel GF 250 μm plates and were visualized with ultraviolet (UV) light (254 nm) and/or KMnO₄ staining. Flash chromatography was performed using normal phase SiliCycle silica gel (40-63D, 60 Å).

Deactivated silica gel was prepared by treating silica gel with EtSiCl₃.¹H, ¹³C and ¹⁹F nuclear magnetic resonance (NMR) chemical shifts are reported in ppm relative to CHCl₃, CH₂Cl₂ and MeOH (i.e. ¹H NMR δ=7.26 and ¹³C NMR=77.0, ¹H NMR=5.32 and ¹³C NMR=54.0, ¹H NMR=3.31 and ¹³C NMR=49.1).

### (rel-1R,8S,9S,4E)-Bicyclo[6.1.0]non-4-ene-9-ylmethanol (5)

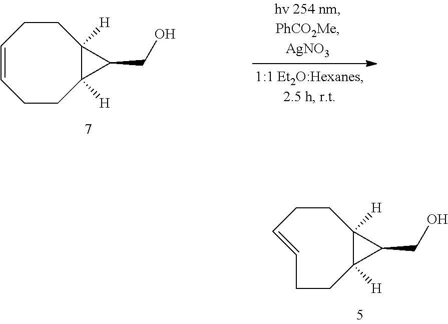

The general photoisomerization procedure was followed using 7 (395 mg, 2.59 mmol) in 1:1 ether/hexanes (250 mL), methyl benzoate (705 mg, 5.18 mmol) and dodecane (491 mg, 2.88 mmol, standard for GC monitoring) in a 250 mL quartz tube. A 50 g Biotage® SNAP column was filled with normal silica gel (2.5 inches) and the remaining space was packed with 10% silver impregnated silica (5.70 g). The column was connected to a pump and flushed with 1:1 ether/hexanes (250 mL). Irradiation was carried out at 254 nm for 2.5 h at which GC monitoring showed no more starting material. The column was flushed with 1:1 ether/hexanes (250 mL) and dried under air flow. The silica was placed into a flask and stirred in ammonium hydroxide (200 mL) and dichloromethane (200 mL) for 10 min. The silica was filtered and washed with additional ammonium hydroxide (100 mL) and dichloromethane (100 mL). The phases were separated and the aqueous layer was extracted an additional three times. The combined organic layers were washed twice with water, dried over Na₂SO₄, filtered and concentrated by rotary evaporation. Purification by column chromatography (25%, EtOAc:Hexanes) to yield 318 mg (2.09 mmol, 81%) of compound 5 as a colorless oil which was stored as a solution in MeOH at −15° C. ¹H NMR (600 MHz, CD₃OD) δ: 5.88 (ddd, J=16.2, 9.3, 6.2 Hz, 1H), 5.16 (dddd, J=16.7, 10.6, 3.9, 1.1 Hz, 1H), 3.50 (d, J=7.7 Hz, 2H), 2.31 (dtd, J=11.4, 3.7, 2.4 Hz, 1H), 2.28 (ddd, J=12.5, 8.4, 6.9 Hz, 1H), 2.21-2.15 (m, 1H), 2.13-2.09 (m, 1H), 1.96-1.86 (m, 2H), 1.20 (dt, J=9.1, 7.7 Hz, 1H), 1.09 (tdd, J=12.9, 11.2, 7.1 Hz, 1H), 0.85-0.71 (m, 2H), 0.60 (dtd, J=13.0, 8.8, 4.6 Hz, 1H), (small peaks attributable to impurities were detected by ¹H NMR at 5.49, 4.09, 2.01, 1.29,1.24 and 0.90 ppm). ¹³C NMR (151 MHz, CD₃OD) δ 139.4, 132.3, 59.5, 35.3, 34.8, 28.6, 28.3, 21.7, 20.2, 19.2; HRMS (EI) [M+H] m/z: calcd for C₁₀H₁₆O: 152.1201; found: 152.1181.

### 2-(2-(2-((syn-(E)-bicyclo[6.1.0]non-4-en-9-yl)methoxy)ethoxy)ethoxy)ethyl 4-methylbenzenesulfonate (8)

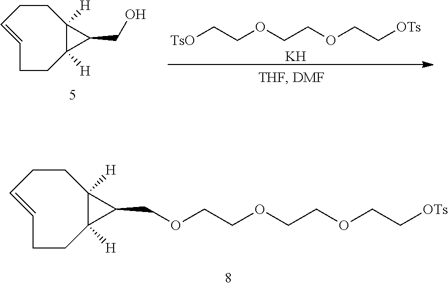

Triethylene glycol bis(p-toluenesulfonate) (972 mg, 2.12 mmol) was added into a flame-dried round bottom flask and dissolved in anhydrous THF (6.0 mL, 0.35M) and DMF (0.6 mL, 3.53M). 5 (100 mg, 0.66 mmol) was added followed by potassium hydride (210 mg, 50% in paraffin, 2.63 mmol). The mixture was stirred at room temperature for 16 h after which saturated aqueous NH₄Cl solution was added followed by ether. The phases were separated and the aqueous layer was extracted an additional three times. The combined organic layers were dried over Na₂SO₄, filtered and concentrated by rotary evaporation. Purification by column chromatography (25-50%, EtOAc:Hexanes) yielded 85 mg (0.19 mmol, 30%) of desired compound 8 as a colorless oil which was stored as a solution in MeOH at −15° C. ¹H NMR (600 MHz, CD₃OD) δ: 7.80 (d, J=8.3 Hz, 2H) 7.45 (d, J=8.4 Hz, 2H), 5.86 (ddd, J=16.2, 9.3, 6.3 Hz, 1H), 5.16 (dddd, J=16.8, 10.6, 3.9, 1.1 Hz, 1H), 4.18-4.12 (m, 2H), 3.68-3.34 (m, 2H), 3.60-3.52 (m, 8H), 3.46-3.41 (m, 2H), 2.46 (s, 3H), 2.34-2.27 (m, 1H), 2.26-2.19 (m, 1H), 2.19-2.11 (m, 1H), 2.11-2.04 (m, 1H), 1.98-1.84 (m, 2H), 1.30-1.19 (m, 1H), 1.14-1.01 (m, 1H), 0.87-0.69 (m, 2H) 0.65-0.55 (m, 1H), (small peaks attributable to impurities were detected by ¹H NMR at 4.63, 4.09, 2.01 and 1.24 ppm); ¹³C APT NMR (100.6 MHz, CD₃OD) δ: 146.5, 139.4, 134.6, 132.4, 131.2, 129.2, 73.1, 71.7, 71.7, 71.6, 71.0, 69.9, 69.1, 35.5, 34.8, 28.8, 28.4, 21.7, 20.3, 19.3, 19.2, (a small peak attributable to dichloromethane was detected by ¹³C at 54.9 ppm); HRMS (LIFDI-TOF) m/z: [M]⁺ Calcd for C₂₃H₃₄O₆S⁺ 438.2076; Found 438.2066.

### syn-(E)-9-((2-(2-(2-fluoroethoxy)ethoxy)ethoxy)methyl)bicycle[6.1.0]non-4-ene (9)

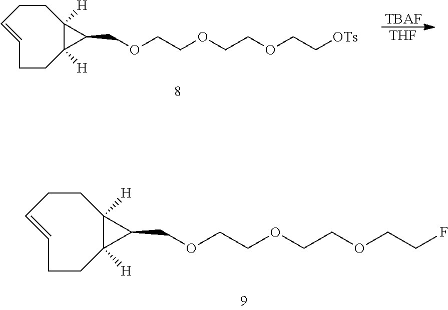

Tosylate 8 (10 mg, 0.02 mmol) was charged into a 4 dram vial and TBAF (0.5 mL, 1.0 M in THF) was added via syringe. The mixture was heated to 60° C. for 3.5 h and subsequently cooled to room temperature. The mixture was diluted with ethyl acetate, washed with saturated aqueous NaHCO₃ and dried over Na₂SO₄. The solution was filtered and concentrated by rotary evaporation. Purification by column chromatography (25%, EtOAc:Hexanes) yielded 5 mg (0.02 mmol, 76%) of 9 as a colorless oil that was stored as a solution in MeOH at −15° C. ¹H NMR (600 MHz, CD₃OD) δ: 5.87 (ddd, J=16.2, 9.3, 6.2 Hz, 1H), 5.17 (ddd, J=14.0, 10.6, 3.8 Hz, 1H), 4.52 (dt, J_{CF}=48 Hz, J_{HH}=4.1 Hz, 2H), 3.72 (dt, J_{CF}=30.1 Hz, J_{HH}=4.0 Hz, 2H), 3.68-3.59 (m, 6H), 3.59-3.54 (m, 2H), 3.44 (d, J=7.5 Hz, 2H), 2.34-2.28 (m, 1H), 2.28-2.21 (m, 1H), 2.19-2.13 (m, 1H), 2.11-2.06 (m, 1H), 1.99-1/84 (m, 2H), 1.27-1.21 (m, 1H), 1.14-1.04 (m, 1H), 0.86-0.80 (m, 1H), 0.79-0.71 (m, 1H), 0.65-0.57 (m, 1H), (small peaks attributable to the cis isomer (5.61 ppm) and an impurity (1.29, 0.90 ppm) were also detected by ¹H NMR); ¹³C APT NMR (100.6 MHz, MeOD) δ: 139.4, 132.4, 84.2 (d, JCF=168 Hz), 71.82, 71.76, 71.74 (d, JCF=20 Hz), 71.65, 71.0, 69.1, 35.5, 34.8, 28.8, 28.4, 20.3, 19.4, 19.3; ¹⁹F NMR (376 MHz, CD₃OD) δ: −224.7 (tt, J=48.1, 30.0 Hz); HRMS (Orbitrap) m/z: [M+Na]⁺ Calcd for C₁₆H₂₇FO₃Na 309.18364; Found 309.18453.

### 3-oxo-1-(4-(6-phenyl-1,2,4,5-tetrazin-3-yl)phenyl)-2,7,10,13,16,19,22,25,28,31,34,37,40-tridecaoxa-4-azatritetracontan-43-oic acid (11)

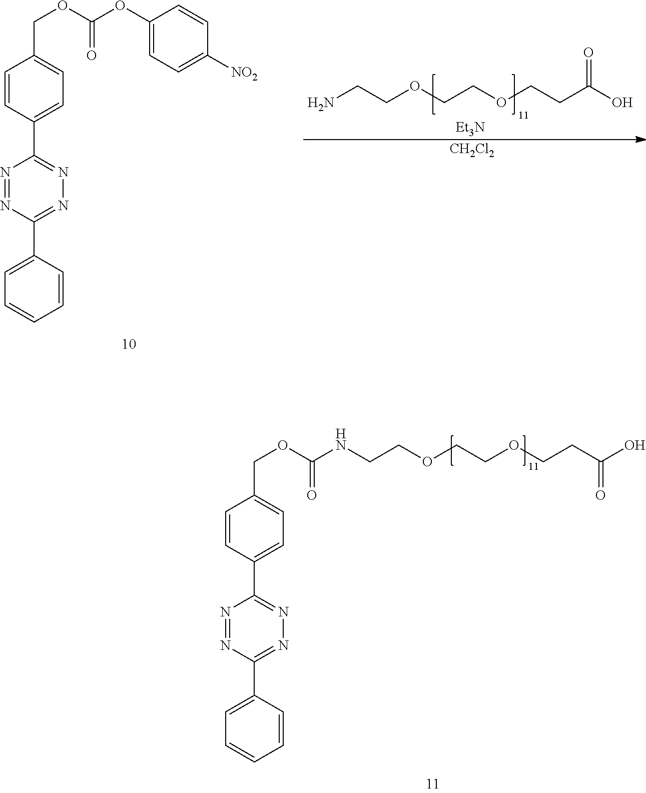

4-nitrophenyl 4-(6-phenyl-1,2,4,5-tetrazin-3-yl)benzyl carbonate (10) (43 mg, 0.10 mmol) and PEG12-Amino acid (31 mg, 0.05 mmol) were dissolved in anhydrous dichloromethane (4.0 mL, 0.01 M). Triethylamine (13.8 μL) was added and the reaction was stirred at room temperature for 30 h. 1N HCl (5 mL) was added and the aqueous phase was extracted with dichloromethane (3×). The combined organics were dried over Na₂SO₄, filtered and concentrated by rotary evaporation. The crude was purified by column chromatography using deactivated silica gel (2.50 g, 0-5% MeOH:DCM) to yield 40 mg (0.04 mmol, 88%) of 11 as a purple solid. mp: 39-40° C.; ¹H NMR (400 MHz, CDCl₃) δ: 8.67-8.61 (m, 4H), 7.70-7.59 (m, 5H), 5.58 (t, J=5.8 Hz, 1H), 5.22(m, 2H), 3.73 (t, J=5.8 Hz, 2H), 3.66-3.54 (m, 48H), 3.39 (q, 5.4 Hz, 2H), 2.60 (bs, 2H); ¹³C NMR (100 MHz, CDCl₃) δ: 173.3, 164.1, 163.8, 156.5, 141.8, 132.9, 131.8, 131.4, 129.5, 128.6, 128.2, 128.1, 70.8, 70.7-70.5 (19 C's), 70.4, 70.3, 70.1, 66.7, 66.0, 41.1, 35.1 (a peak attributed to CH₂Cl₂was observed at 54 ppm); HRMS (LIFDI-TOF) m/z: [M+Na]⁺ Calcd for C₄₃H₆₅N₅O₁₆Na 930.4324; Found 930.4336.

### 2,5-dioxopyrrolidin-1-yl-3-oxo-1-(4-(6-phenyl-1,2,4,5-tetrazin-3-yl)phenyl)-2,7,10,13,16,19,22,25,28,31,34,37,40-tridecaoxa-4-azatritetracontan-43-oate (11a)

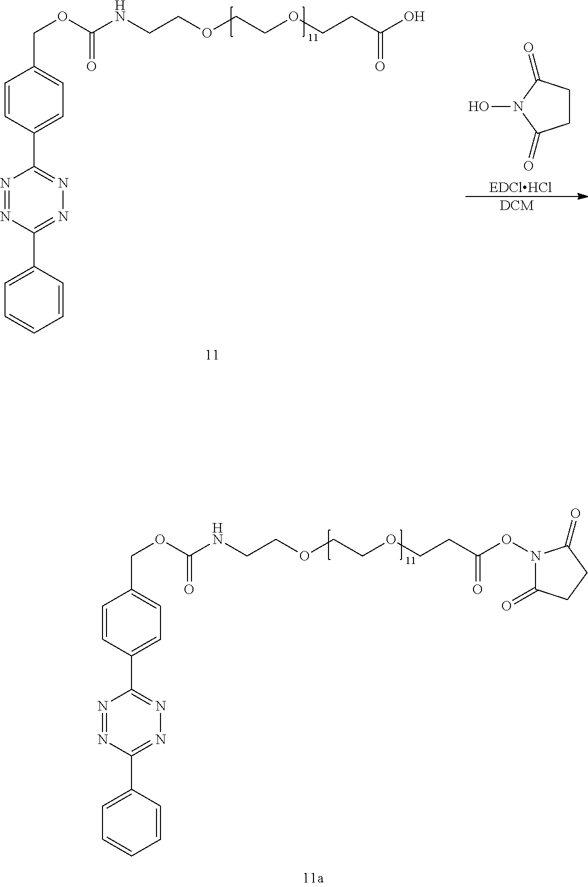

Acid 11 (24 mg, 0.0264 mmol), N-hydroxysuccinimide (NHS) (5.0 mg, 0.0434 mmol) and 1-ethyl-3-(3-dimethylaminopropyl)carbodiimide hydrochloride (EDCI) (8.0 mg, 0.0417 mmol) were added to a flame-dried round bottom flask. The mixture was dissolved in anhydrous dichloromethane (2.0 mL, 0.02 M) and stirred at room temperature for 16 h. The solution was directly applied to a column of deactivated silica gel (2.50 g) and washed with large amounts of dichloromethane after which product was eluted with 5% MeOH:DCM. Further purification using HILIC (2.50 g silica gel, 5% H₂O:MeOH) yielded 19 mg (0.02 mmol, 72%) of 11a as a purple solid. mp: 37-39° C.; ¹H NMR (400 MHz, CD₂Cl₂) δ: 8.67-8.60 (m, 4H), 7.70-7.59 (m, 5H), 5.54 (bs, 1H, NH), 5.22 (s, 2H), 3.83 (t, J=6.3 Hz, 2H), 3.67-3.52 (m, 48H), 3.39 (m, 2H), 2.88 (t, J=6.3 Hz, 2H), 2.84-2.76 (bs, 4H); ¹³C NMR (100 MHz, CD₂Cl₂) δ: 169.5, 167.3, 164.4, 164.2, 156.5, 142.5, 133.0, 132.3, 131.8, 129.7, 128.7, 128.3, 128.2, 71.0, 70.9-70.7 (20 C's), 70.3, 66.0, 41.4, 32.5, 26.0; HRMS (LIFDI-TOF) m/z: [M+Na]⁺ Calcd for C₄₇H₆₈N₆O₁₈Na 1027.4488; Found 1027.4487, : [M+K]⁺ Calcd for C₄₇H₆₈N₆O₁₈K 1043.4227, Found 1043.4200.

RGDyK-Tz (12)

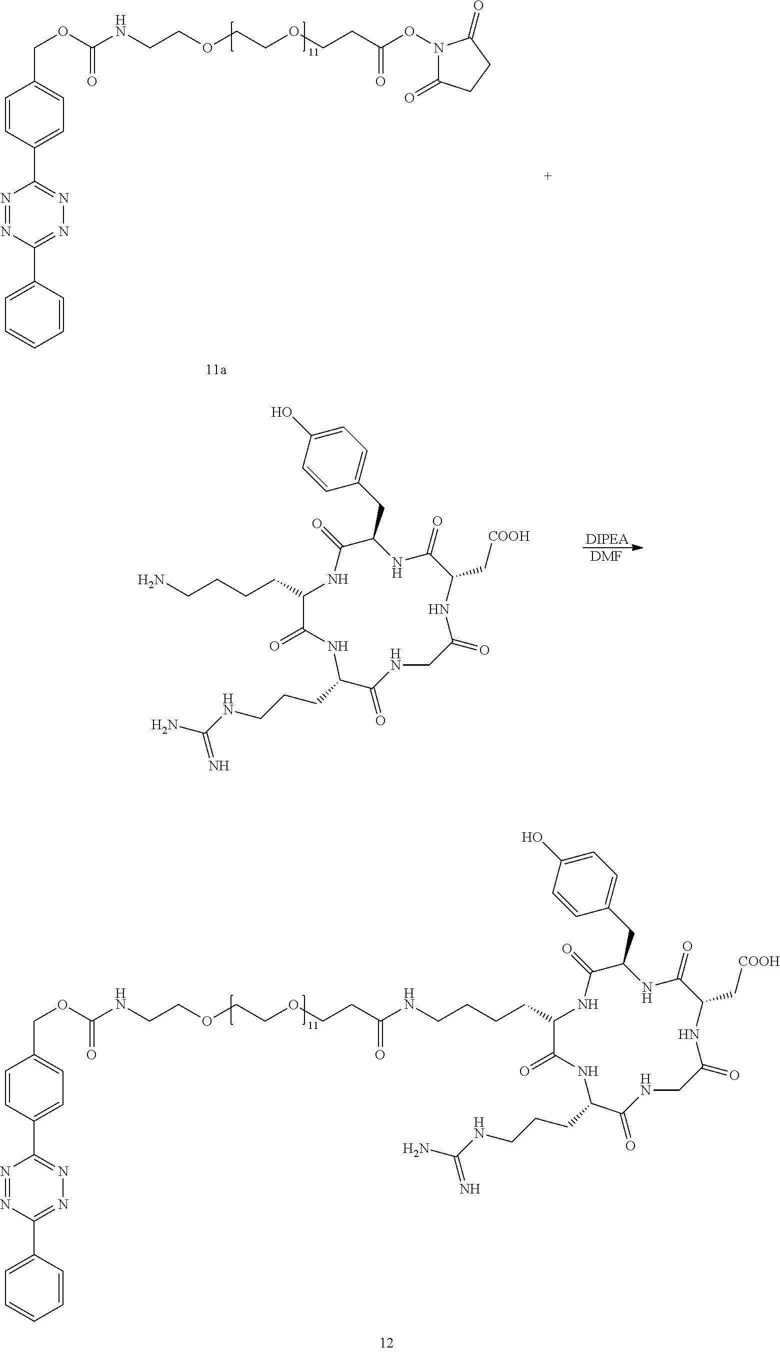

RGDyK (3.0 mg, 0.0048 mmol) was added to a 4 dram vial followed by TzPEG12NHS (11a) (10 mg, 0.0099 mmol) as a solution in anhydrous dimethylformamide (400 μL, 0.01 M). N,N-diisopropylethylamine (3.0 mg, 0.02 mmol) was added as a solution in anhydrous dimethylformamide (100 μL, 0.20 M) and the reaction was allowed to stir at room temperature for 18 h. H₂O was added and the solvents were removed via freeze drying. The residue was purified by RP HPLC to yield 7.2 mg (12) (0.005 mmol, 99%) as a pink solid. HRMS (LIFDI-TOF) m/z [M+Na]⁺ Calcd for C₇₀H₁₀₄N₁₄O₂₃Na 1531.7296; Found 1531.7279.

RGDvK-Tz-sTCOPEGF (15)

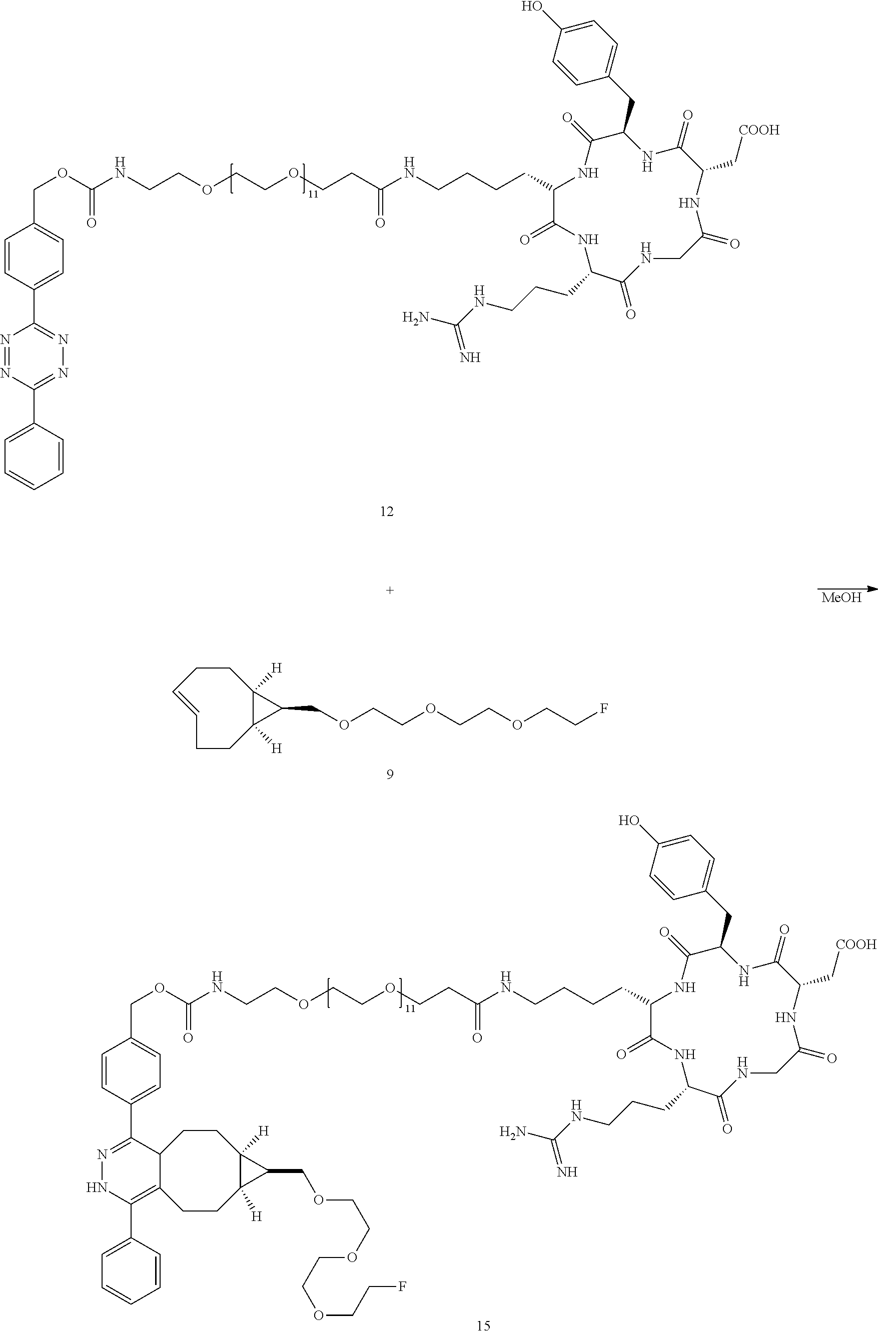

Tetrazine-RGD conjugate (12) (0.3 mg, 0.0002 mmol) was dissolved in methanol (0.5 mL) and sTCOPEGF (9) (23 μL of a 2.5 mg/mL solution in MeOH, 0.06 mg, 0.0002 mmol) was added dropwise. The reaction was monitored by UV/Vis and was complete within 1 min. The product (15) was purified by reverse phase HPLC (C-18 column, 10% ACN+0.1% formic acid to 100% ACN+0.1% formic acid).

General Procedure for Stop-Flow Kinetic Analysis of sTCO's and 11 at Variable Concentrations

The reaction between sTCOs 4 & 5 and the PEGylated tetrazine 11 was measured under pseudo-first order conditions in water:methanol 45:55 by following the exponential decay of the tetrazine at 298 nm over time using an SX 18MV-R stopped flow spectrophotometer (Applied Photophysics Ltd.). Solutions were prepared for the sTCO concentrations see table below water:methanol 45:55) and the tetrazine (0.1 mM in water:methanol 45:55) and thermostatted in the syringes of the spectrophotometer before measuring. An equal volume of each was mixed by the stopped flow device (resulting concentrations shown in the table below). 400 data points were recorded over a period of 1 second, and performed in sextuplicate at 298 K. The k_{obs} was determined by nonlinear regression analysis of the data points using Prism software (v. 6.00, GraphPad Software Inc.).

TABLE 1
Rate constants for the reaction of trans-cyclooctenes (sTCO's 4 & 5) with

| | Resulting | Initial | Resulting | | | |
|---|---|---|---|---|---|---|
| | concentration | concentration | concentration | | | Mean |
| | Tetrazine | sTCO | sTCO | | k₂ | k₂ |
| | [mM] | [mM] | [mM] | k_{obs} | [M⁻¹s⁻¹] | [M⁻¹s⁻¹] |
| syn | 0.05 | 0.49 | 0.245 | 8.415 | 34347 | 36,100 |
| sTCO | | 0.98 | 0.49 | 18.41 | 37571 | +/− |
| | | 1.96 | 0.98 | 35.08 | 35796 | 1,400 |
| | | 2.94 | 1.47 | 54.18 | 36857 | |
| anti | | 0.5 | 0.25 | 7.458 | 29832 | 31,700 |
| sTCO | | 1.0 | 0.5 | 15.84 | 31680 | +/− |
| | | 2.0 | 1 | 32.51 | 32510 | 1,300 |
| | | 3.0 | 1.5 | 48.98 | 32653 | |

PEGylated tetrazine 11 at 25° C. in water:methanol (45:55) measured under pseudo first order conditions using SX 18MV-R stopped flow spectrophotometer. Values were determined from an average of four runs.
   Synthesis and Characterization of a Conformationally Strained Trans-Cyclooctene with a Cis-Fused Cyclopentane Ring

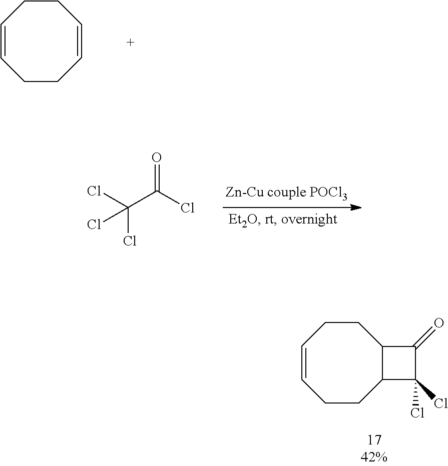

A 100 mL 2 neck round-bottom flask was flame dried under vacuum, then charged with nitrogen. Cyclooctadiene (20 mmol, 2.1 gram) in about 20 mL dry ether was added to the flask via syringe. Zinc-copper couple (30 mmol, 2.0 gram, 1.5 equiv) was then added to the ether solution under nitrogen. The suspension was stirred at room temperature. Trichloroacetic chloride (25 mmol, 4.5 gram, 1.25 equiv) and phosphorus (V) oxychloride (25 mmol, 3.8 gram, 1.25 equiv) were dissolved in approximately 10 mL dry ether, and added dropwise to the stirring suspension via an additional funnel over an hour. The ether solution refluxed mildly after the addition. The reaction was allowed to run overnight, it was then filtered through a celite pad by vacuum filtration. A dark brown solution was resulted. Solvent was removed by rotary evaporator. The residue was first extracted with hexane, followed by ether/hexane (3/1). The organic solution was combined, and then washed sequentially with water, saturated sodium bicarbonate and brine. The resulted organic solution was dried by sodium sulfate. The drying agent was removed by gravity filtration and the solution was concentrated by rotary evaporator. A viscous yellow liquid was obtained. This material was subject to Kugelrohr distillation (0.15 mmHg, 130° C.) to yield 1.7 gram slightly yellow liquid (42%) as product.

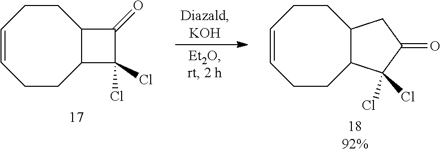

A simplified diazomethane preparation apparatus was used in this step. An Erlenmeyer flask (B) was charged with dichloroketone 17 (20 mmol, 2.2 gram) in about 30 mL ether. Diazald (60 mmol, 12.8 gram, 3 equiv) was dissolved with stirring in approximately 100 mL methanol in a . vacuum filtration flask until a clear yellow solution was formed. A stream of nitrogen was then allowed to pass through the whole system. Potassium hydroxide (200 mmol, 11.2 gram, 10 equiv) was dissolved with a minimal amount of water and added dropwise to the Diazald solution at intervals through a rubber septum via a syringe. Yellow diazomethane was generated and was carried into the Erlenmeyer flask by a nitrogen flow. Potassium hydroxide solution was added continuously till the yellow color in the vacuum filtration flask was discharged, after which stirring was continued for one hour. A small amount of glacial acetic acid was then added to the Erlenmeyer flask to quench any unreacted diazomethane. The ether solution in the Erlenmeyer flask was then transferred to a separation funnel. It was washed sequentially with water, saturated sodium bicarbonate and brine. The resulted organic solution was dried by sodium sulfate. The drying agent was removed by gravity filtration and the solution was concentrated by rotary evaporator. 2.2 gram of a slightly yellow liquid was obtained as product (92%).

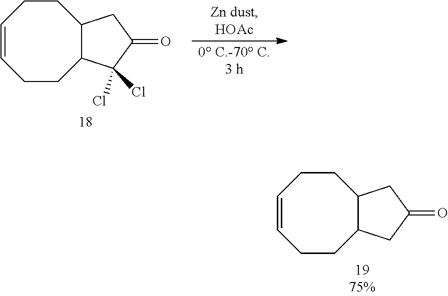

Dichloroketone 18 (9.3 mmol, 2.2 gram) was dissolved with about 15 mL glacial acetic acid. This solution was added dropwise to a suspension of zinc dust (47 mmol, 3.1 gram, 5 equiv) in approximately 15 mL glacial acetic acid which was cooled down to 0° C. Ice-bath was removed once the addition was finished, and the reaction mixture was heated to 70° C. After reacting at 70° C. for 3 hours, the reaction mixture was allowed to cool down to ambient temperature, then it was diluted with ether. The reaction mixture was transferred to a separation funnel, and it was washed sequentially with water, saturated sodium bicarbonate and brine. The resulted organic solution was dried by sodium sulfate. The drying agent was removed by gravity filtration and the solution was concentrated by rotary evaporator. The residue was further purified by silica gel chromatography using hexane/ethyl acetate (4/1) as eluent. 1.1 gram of a clear, colorless liquid was obtained as the expected ketone product 19 (75%).

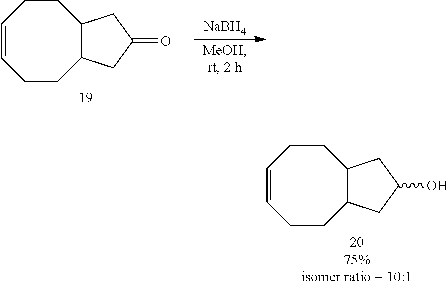 [SMILES: O=C1CC2CC/C=C\CCC2C1 ; "NaBH4" ; OC1CC2CC/C=C\CCC2C1]

Bicyclic ketone 19 (5.6 mmol, 0.92 gram) was dissolved with about 20 mL methanol in a 100 mL round-bottom flask. Sodium boron hydride (23 mmol, 0.88 gram, 4 equiv) was added to the solution. Copious bubbles were produced instantly. The reaction was allowed to run at ambient temperature for 2 hours, it was then quenched by addition of water. The reaction mixture was transferred to a separation funnel, and it was extracted with 25 mL dichloromethane 3 times. The dichloromethane solution was combined, and it was washed sequentially with water, saturated sodium bicarbonate and brine. The resulted organic solution was dried by sodium sulfate. The drying agent was removed by gravity filtration and the solution was concentrated by rotary evaporator. The residue was further purified by silica gel chromatography using hexane/ethyl acetate (4/1) as eluent. 0.75 gram of a clear, colorless liquid was obtained as the expected alcohol product 4 (75%) NMR of compound 20 revealed that it contained both syn and anti diastereomers in 10/1 ratio.

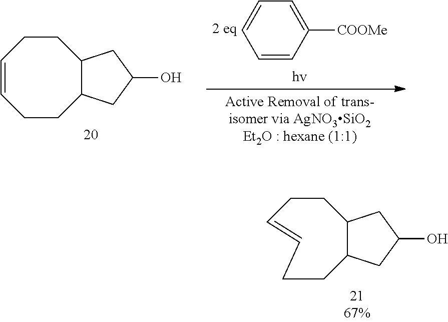 [SMILES: OC1CC2CC/C=C\CCC2C1 ; COC(=O)c1ccccc1 ; OC1CC2CC/C=C/CCC2C1]

The continuous flow apparatus described in Royzen, M.; Yap, G. P.; Fox, J. M. J. Am. Chem. Soc. 2008, 130, 3760 was used for the photoisomerization. 100 g Biotage SNAP cartridge (Biotage part no. FSK0-1107-0050) was used to house the silica gel and AgNO₃ impregnated silica gel. The SNAP cartridge that contained a bed of unmodified silica gel was topped with 17 g of silica gel which was impregnated with AgNO₃ (1.7 g, 10 mmol). (Z)-2,3,3a,4,5,8,9,9a-octahydro-1H-cyclopenta[8]annulen-2-ol 20 (1.1 g, 6.6 mmol) and methyl benzoate (1.8 g, 13 mmol) were placed in a quartz flask and dissolved in 400 mL of 1:1 Et₂O:hexanes. The solution was equilibrated through the continuous flow system at a 100 mL/min flow rate and simultaneously degassed with nitrogen for 15 minutes. The solution in the quartz flask was then irradiated (254 nm) under continuous flow conditions (100 mL/min) for 6 hours, at which point GC analysis indicated that the reaction was complete. The SNAP cartridges were flushed with 400 mL of 1:1 Et₂O/hexanes and then dried with compressed air. The dried silica gel was transferred to a 1 L Erlenmeyer flask. Concentrated aqueous NH₄OH (400 mL) and methylene chloride (400 mL) were sequentially added to the flask, and the resulting biphasic mixture filtered. The filter cake was washed with additional methylene chloride (100 mL) and ammonium hydroxide (100 mL). The filtrate was transferred to a separatory funnel and partitioned. The aqueous layer was extracted twice with methylene chloride. The organic layers were combined, washed twice with water then dried with magnesium sulfate, filtered, and concentrated using a rotary evaporator. Column chromatography (1:2 Et₂O:hexanes) afforded 0.74 g of 21 (67%) as a colorless oil. Compound 21 became a white solid upon storage in a freezer.

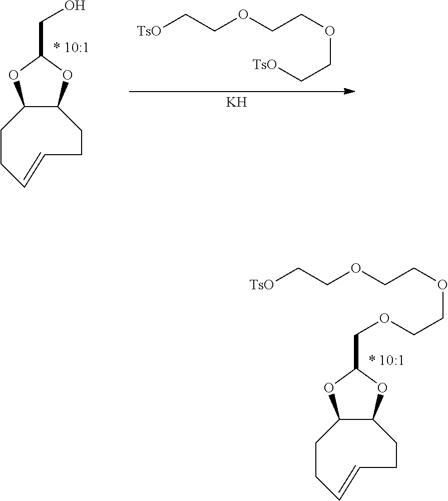

Synthesis of dTCO-Ts:

To a solution of KH (0.200 g, 5 mmol) in dry THF/DMF (25 mL/2.5 mL), were added bis-peg tosylate (2.14 g, 4.67 mmol) and dTCO-alcohol (0.200 g, 1.31 mmol) at RT. After 14 h, the resulting mixture was quenched with sat. NH₄Cl (20 mL) at 0° C. and The resulting solution was extracted with diethyl ether (3×150 mL), washed with water (2×150 mL), over Na₂SO₄, concentrated and purified by column chromatography using 0-70% acetone in hexane as an eluent to give dTCO-Ts (0.192 g, 31%) as clear.

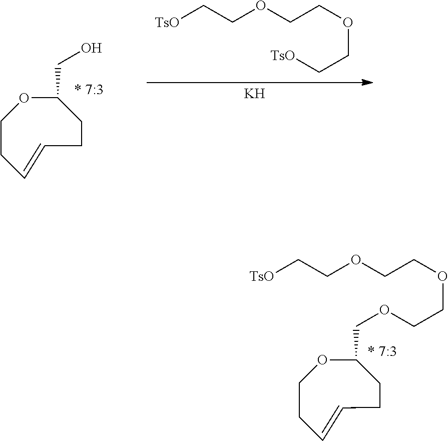

Synthesis of Oxo-TCO-Ts:

To a solution of KH (0.05 g, 1.22 mmol) in dry THF/DMF (8 mL/1 mL), were added bis-peg tosylate (0.481 g, 1.05 mmol) and Oxo-TCO-alcohol (0.05 g, 0.35 mmol) at RT. After 14 h, the resulting mixture was quenched with sat. NH₄Cl (10 ml) at 0° C. and The resulting solution was extracted with diethyl ether (3×50 mL), washed with water (2×50 mL), dried over Na₂SO₄, concentrated and purified by column chromatography using 0 to 70% acetone in hexane as an eluent to give Oxo-TCO-Ts (0.06 g, 40%) as clear oil.

Preparation of ¹⁹F Labeled Strained Trans-Cyclooctenes

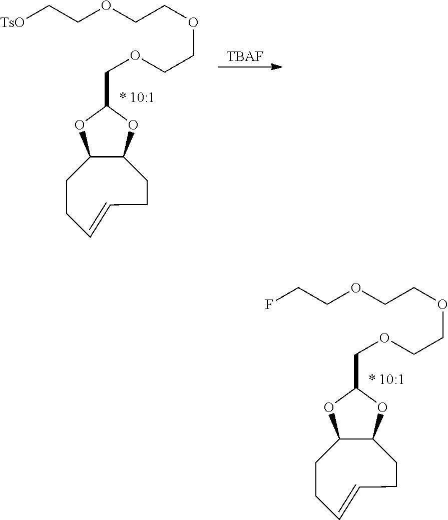

Synthesis of dTCO-¹⁹F:

TABF (1M in THF) was added to the sample vial containing dTCO-Ts (0.015 g, 0.0319 mmol) at rt. After 3 h, the reaction mixture was diluted with EtOAc and all the solvents were evaporated. To the resulting residue, was added EtOAc and sat. NH₄Cl. Two layers were separated and the organic layer was washed with water, dried with Na₂SO4, filtered and purified by column chromatography by using 0-100% EtOAc in Hexane as an eluent to give dTCO-¹⁹F (0.09 g, 88.6%) as colorless clear oil

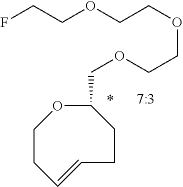

Synthesis of Oxo-TCO-¹⁹F:

TABF (1M in THF) was added to the sample vial containing dTCO-Ts (0.015 mg, 0.032 mmol) at rt. After 3 h, the reaction mixture was diluted with EtOAc and all the solvents were evaporated. To the resulting residue, was added EtOAc and sat. NH₄Cl. Two layers were separated and the organic layer was washed with water, dried with Na₂SO4, filtered and purified by column chromatography by using 0-100% EtOAc in Hexane as an eluent to give dTCO-¹⁹F (0.08 g, 90.5%) as colorless clear oil. The ¹⁸F analog can be made analogously.

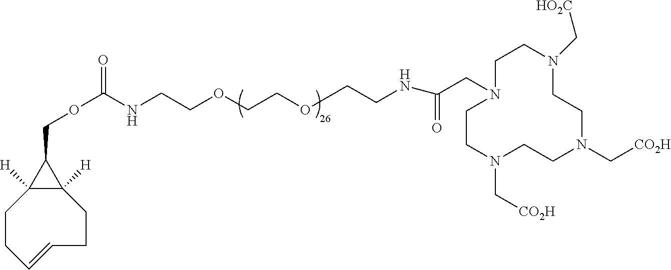

Synthesis of sTCO-DOTA:

To a solution of DIPEA (0.034 mL, 0.196 mmol) and DOTANHS (0.015 mg, 0.0196 mmol) in DMF (1.5 mL) was added (PEG)₂₆ diamine (0.024 g, 0.0196 mmol) in DMF (1.5 mL) drop wise over 1 hr. After 14 h of stirring, sTCO carbamate (0.0062 mg, 0.0196) was added. The resulting reaction mixture was stirred for additional 6 h. All the solvents were evaporated, and purified by using Yamazen C18 (7+14 g, as shown in Fig below) column chromatography 0 to 100% MeOH in H₂O as an eluent to give sTCO-DOTA (0.0089 g, 28%) as a clear oil and it was stored in methanol at −20° C.

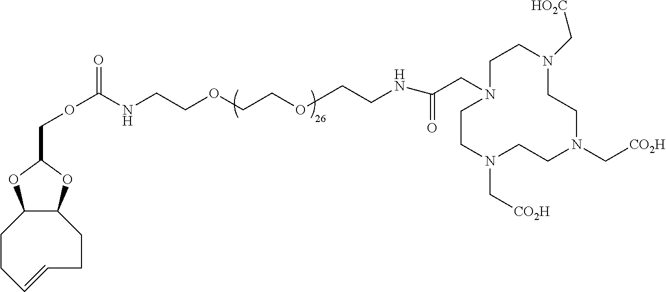

Synthesis of dTCO-DOTA:

To a solution of DIPEA (0.034 mL, 0.196 mmol) and DOTANHS (0.015 mg, 0.0196 mmol) in DMF (1.5 mL) was added (PEG)₂₆diamine (0.024 g, 0.0196 mmol) in DMF (1.5 mL) drop wise over 1 hr. After 14 hrs of stirring, dTCO carbamate (0.0068 mg, 0.0196) was added. The resulting reaction mixture was stirred for additional 6 hrs. All the solvents were evaporated, and purified by using Yamazen C18 (7+14 g) column chromatography 0 to 100% MeOH in H₂O as an eluent to give sTCO-DOTA (0.0079 g, 22%) as a clear oil and it was stored in methanol at −20° C.

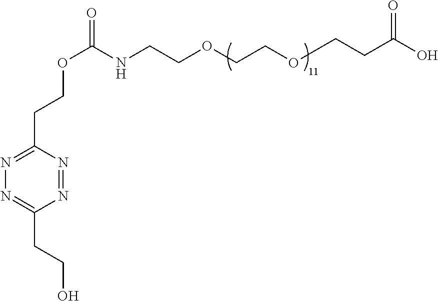

Synthesis of diolTz-acid:

To the solution of Peg₁₂aminoacid (0.082 g, 0.13 mmol) and triethylamine (0.18 mL, 1.3 mmol) in dichloromethane (4 mL) was added diolTz-p-nitrophenylcarbamate (0.045 g, 0.13 mmol) as a solution in dichloromethane (2 mL) over 2 hrs. After 24 h, was added dichloromethane and 1N HCl. Organic layer was separated, dried with MgSO4, purified with deactivated silica using 2-10% methanol in dichloromethane afforded the required diolTz-acid (65 mg, 61.5%) as a pink solid.

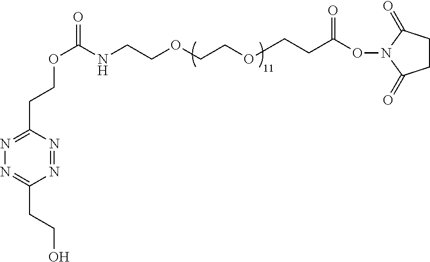

Synthesis of diolTz-NHS:

N-Hydroxysuccinimide (0.0034 g, 0.029 mmol) and N-(3-Dimethylaminopropyl)-N′-ethylcarbodiimide hydrochloride (0.0054 g, 0.028 mmol) were added to a flask containing a solution of diolTz-acid (0.015 mg, 0.018) in dichloromethane (2 mL). After stirring for 24 h, the resulting solution directly purified using deactivated silica with 0-5% methanol in dichloromethane to give diolTz-NHS (0.010, 63%) as a pink oil.

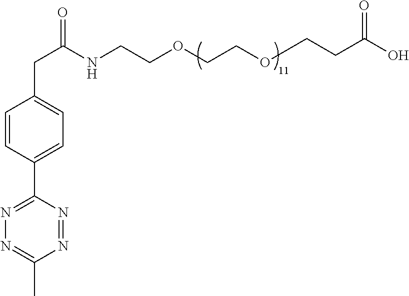

Synthesis of mePhTz-Acid:

To a solution of mePhTz-NHS (0.032 g, 0.0956) and Peg₁₂aminoacid (0.060 g, 0.0956) in DMF/DCM 4 ml/2 ml was added di-isopropylethylamine (0.034 mL, 0.18). After 14 hrs, solvents were evaporated and re-dissolved the residue in DCM and 1N HCl. Two layers were separated and the organic layer was dried over MgSO4, concentrated, purified with deactivated silica with 0-5% methanol in dichloromethane to give mePh-Tz-acid (0.072 g, 90%) as a pink solid.

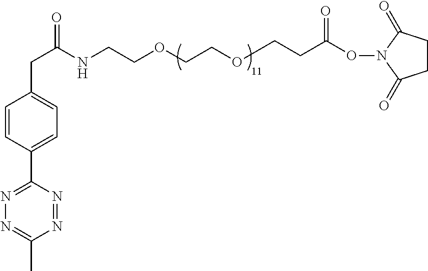

Synthesis of mePhTz-NHS:

N-Hydroxysuccinimide (0.0093 g, 0.0817 mmol) and N-(3-Dimethylaminopropyl)-N′-ethylcarbodiimide hydrochloride (0.0148 g, 0.0778 mmol) were added to a flask containing a solution of tetrazine acid (0.036 mg, 0.043) in DCM (2 mL). After stirring for 14 hrs, the resulting solution directly purified using deactivated silica with 0-5% methanol in dichloromethane to give mePhTz-NHS (0.035 g, 87%) as a pink oil.

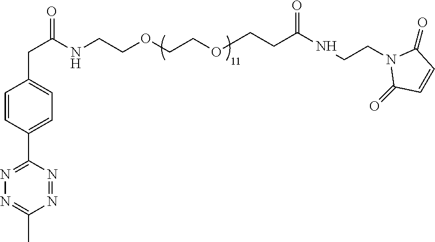

Synthesis of mePhTz-maleimide:

To a solution of mePhTz-NHS (0.037 mg, 0.039 mmol) and N(2-aminoethylmaleimide TFA salt) in DMF (1 mL) under nitrogen was and DIPEA (0.0224 mL, 0.129 mmol). After stirring the reaction mixture for 24 h, all the solvents were evaporated and purified using deactivated silica with 1-5% methanol in dichloromethane to give (0.006 mg, 18%) as a pink oil.

Synthesis of diolTz-NT:

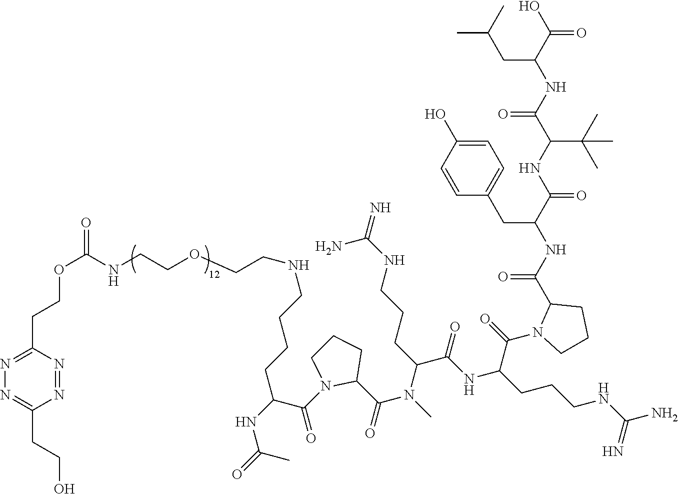

To a solution of dioltzNHS (0.0021 g, 0.00230 mmol) and Neurotensin amine (0.001 g, 0.001153 mmol) in DMF (0.5 mL) was added diisopropylethylamine (0.001 mL, 0.00230 mmol). After 48 h solvents were evaporated and purified using Yamagen C18 column with 0-100% methanol: water gave titled product (0.009 mg, 41%) as light pink oil.

### 4-(Di(but-3-en-1-yl)(methyl)silyl)butanenitrile

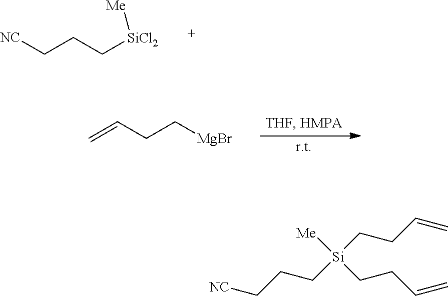

A dry round-bottomed flask was charged with Mg (1.24 g, 51.7 mmol, 3.00 equiv) and dry THF (125 ml) under nitrogen atmosphere. 4-Bromo-1-butene (5.60 mL, 55.2 mmol, 3.21 equiv) was introduced to the flask dropwise via syringe. The reaction mixture was allowed to stir at room temperature. After the formation of the Grignard reagent was judged complete, HMPA (15.0 mL, 86.0 mmol, 5.00 equiv) was added, followed by 4-(dichloro(methyl)silyl)butanenitrile (2.70 ml, 17.2 mmol, 1.00 equiv). The reaction mixture was stirred at room temperature overnight. After reaction, THF was removed via rotary evaporation. Saturated aq. NH₄Cl (80 mL) and ethyl acetate (80 mL) were added and the aqueous layer was extracted three times with ethyl acetate. The organics were combined, dried with anhydrous MgSO₄, filtered, and concentrated via rotary evaporation. Purification by flash column chromatography (1% diethyl ether/hexane) afforded the title compound as colorless oil (2.14 g, 9.66 mmol, 56% yield).

### (Z)-Si-(3-Cyanopropyl)-Si-methyl-5-sila-cycloheptene

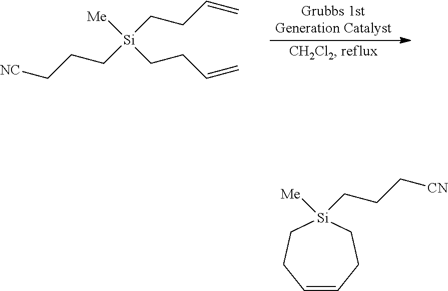

4-(Di(but-3-en-1-yl)(methyl)silyl)butanenitrile (400 mg, 1.81 mmol, 1.00 equiv) was dissolved in dry CH₂Cl₂ (120 ml). Grubbs' 1^{st} generation catalyst (74.3 mg, 0.0903 mmol, 0.0500 equiv) was added as a solution in dry CH₂Cl₂ (37 mL) and the solution was heated to reflux for 5 hours. After cooling to room temperature, the reaction mixture was concentrated via rotary evaporation. Purification by flash column chromatography (1% diethyl ether/hexane) afforded the title compound (299 mg, 1.55 mmol, 85% yield) as colorless oil.

### (Z)-Si-(4-Oxobutyl)-Si-methyl-5-silacycloheptene

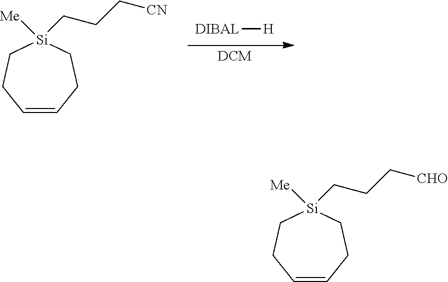

A round-bottomed flask was charged with a solution of (Z)-Si-(3-Cyanopropyl)-Si-methyl-5-silacycloheptene (656 mg, 3.39 mmol, 1.00 equiv) in CH₂Cl₂ (4.5 mL) under an atmosphere of nitrogen. The flask was cooled by a bath of dry ice/acetone (−78° C.), and DIBAL-H (4.1 mL of a 1.0 M solution in CH₂Cl₂, 4.1 mmol, 1.2 equiv) was slowly added via syringe. The dry ice/acetone bath was then replaced with a −40° C. bath (dry ice/acetonitrile), and stirring was continued for 1 hour. The cold bath was then replaced by an ice bath (0° C.). At 0° C., H₂O (0.14 mL) and 15% NaOH (0.14 mL) were sequentially added dropwise. Additional water (0.34 mL) was added, and the ice bath was removed and the mixture allowed to stir for 15 min at r.t. Some anhydrous magnesium sulfate was added and stir for another 15 min. The mixture was filtered to remove solids, which were rinsed with excess dichloromethane. The dichloromethane solutions were combined and concentrated. Purification by flash column chromatography (15% diethyl ether/hexane, R_{f}=0.48) afforded the title compound (422 mg, 2.15 mmol, 63% yield) as colorless oil.

### (Z)-Si-(4-Hydroxybutyl)-Si-methyl-5-silacycloheptene:

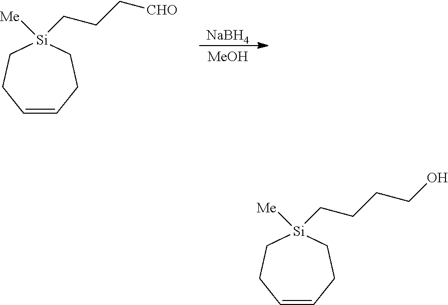 [SMILES: "MeOH" ; C[Si]1(CCCC=O)CCC=CCC1 ; "NaBH4" ; C[Si]1(CCCCO)CCC=CCC1]

A 25 mL round-bottomed flask was charged with (Z)-Si-(4-Oxobutyl)-Si-methyl-5-silacycloheptene (422 mg, 2.15 mmol, 1.00 equiv) and methanol (11 mL). The flask was cooled by an ice bath (0° C.), and the mixture was magnetically stirred. Sodium borohydride (81.3 mg, 2.15 mmol, 1.00 equiv) was added slowly in small portions as a solid to the reaction mixture. The ice bath was removed, and the mixture was allowed to stir while warming to r.t. for 1 h. Water (3 mL) and 3M HCl (3 mL) were sequentially and cautiously added dropwise to the mixture. Methanol was removed by rotary evaporation, and the remainder was thrice extracted with diethyl ether. The combined organics were dried with anhydrous Na₂SO₄, filtered, and concentrated. Purification by flash column chromatography (5%-10% diethyl ether/hexane) afforded the title compound (403 mg, 2.03 mmol, 95% yield) as a colorless oil.

### (E)-Si-(4-Hydroxybutyl)-Si-methyl-5-silacycloheptene

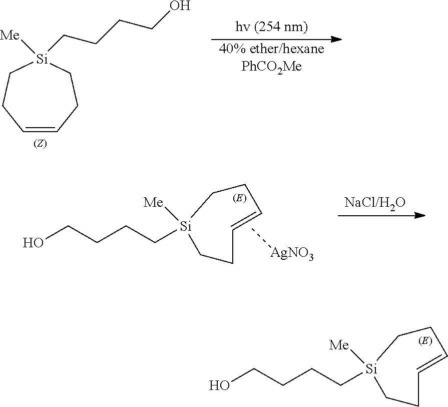

(Z)-Si-(4-Hydroxybutyl)-Si-Methyl-5-silacycloheptene (100 mg, 0.510 mmol, 1.00 equiv) and methyl benzoate (138 mg, 1.02 mmol, 2.00 equiv) were placed in a quartz flask and dissolved in 100 mL of 2:3 Et₂O:hexanes that had been degassed through three freeze/pump/thaw cycles. Dodecane (86 mg, 0.51 mmol, 1.0 equiv) was added to the flask to allow for GC monitoring. The solution in the quartz flask was then irradiated (254 nm) under continuous flow conditions (100 mL/min) for 3 hours with N₂ sparging, at which point GC analysis indicated that the reaction was complete. The SNAP cartridge was flushed with 200 mL of 1:4 Et₂O/hexanes and then dried with compressed air. The SNAP cartridge was then flushed with 225 mL of EtOH to afford an ethanol solution of (E)-Si-(4-Hydroxybutyl)-Si-methyl-5-silacycloheptene.AgNO₃. The ethanol solution was concentrated via rotary evaporation, affording a tan viscous oil consisting of trans-cycloheptene.AgNO₃ complex (0.377 mmol by NMR analysis, 74% yield) and free AgNO₃. To a solution of (E)-Si-(4-Hydroxybutyl)-Si-Methyl-5-silacycloheptene.AgNO₃(51.0 mg in ethanol, 0.19 mmol, 1.0 equiv) was added CH₂Cl₂ (5 mL) followed by saturated brine (5 mL). The aqueous layer was extracted with CH₂Cl₂ (2×5 mL). The organics were dried with anhydrous MgSO₄ and filtered to provide a solution of silver-free (E)-Si-(4-Hydroxybutyl)-Si-methyl-5-silacycloheptene. (Z)-10,10-dichlorobicyclo[6.2.0]dec-4-en-9-one was prepared following the procedure described in Org. Syn. Coll. 1993, 8, 377. Reactions were monitored by thin layer chromatography (TLC) performed on SiliCycle silica gel GF 250 μm plates and were visualized with ultraviolet (UV) light (254 nm) and/or p-Anisaldehyde staining. Flash chromatography was performed using normal phase SiliCycle silica gel (40-63D, 60 Å).

### (Z)-bicyclo[6.2.0]dec-4-en-9-one (30)

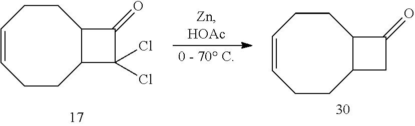 [SMILES: O=C1C2CC/C=C\CCC2C1(Cl)Cl ; O=C1CC2CC/C=C\CCC12]

A solution of zinc dust (520 mg, 7.95 mmol) was added into a flame-dried two-neck round bottom flask and suspended in acetic acid (4.0 mL, 2.0 M). The flask was cooled to 0° C. and 17 (450 mg, 2.10 mmol) was added dropwise as a suspension in acetic acid (4.0 mL, 0.5 M). After the addition was complete, the ice bath was removed and the reaction was heated to 70° C. for 2 hrs. Ether was added to the flask and the solution was transferred to a separatory funnel containing ice water. The organic phase was extracted twice with cold water. The organic layers were then combined and washed three times with saturated aqueous NaHCO₃ and twice with brine. The organic layers were dried over Na₂SO₄, filtered and concentrated by rotary evaporation to yield 300 mg (1.99 mmol, 95%) of 30 with no further purification.

### (Z)-bicyclo[6.2.0]dec-4-en-9-ol (31)

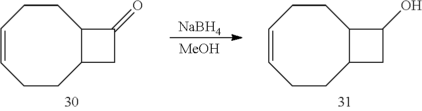 [SMILES: "MeOH" ; O=C1CC2CC/C=C\CCC12 ; "NaBH4" ; OC1CC2CC/C=C\CCC12]

30 (300 mg, 2.0 mmol) was added into a flame-dried round bottom flask and dissolved in anhydrous MeOH (4.0 mL, 0.5 M). Sodium borohydride (303 mg, 8.0 mmol) was added and the reaction was allowed to stir at r.t. for 1 hr. The reaction was cooled to 0° C., quenched with water and extracted three times with dichloromethane. The organic phase was then washed three times with saturated aqueous NaHCO₃ and twice with brine. The combined organic layers were dried over Na₂SO₄, filtered and concentrated by rotary evaporation. Purification by column chromatography (1:7, EtOAc:Hexanes) yielded 107 mg (0.70 mmol, 36%) of 31 as a mixture of diastereomers.

### (E)-bicyclo[6.2.0]dec-4-en-9-ol (4)

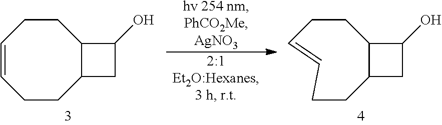 [SMILES: OC1CC2CC/C=C\CCC12 ; "2:1 Et2O:Hexanes,3 h, r.t." ; "hv 254 nm,PhCO2Me,AgNO3" ; OC1CC2CC/C=C/CCC12]

The general photoisomerization procedure described in J. Am. Chem. Soc. 2008, 130, 3760 was followed, using 3 (50 mg, 0.33 mmol) in 2:1 ether/hexanes (100 mL), methyl benzoate (90 mg, 0.67 mmol) and dodecane (85 mg, 0.50 mmol, standard for GC monitoring) in a 150 mL quartz tube. A 10 g Biotage® SNAP column was filled with normal silica gel (2.5 inches) and the remaining space was packed with 10% silver impregnated silica (0.80 g). The column was connected to a pump and flushed with 2:1 ether/hexanes (100 mL). Irradiation was carried out at 254 nm for 3 hr at which GC monitoring showed no more starting material. The column was flushed with 2:1 ether/hexanes (100 mL) and dried under air flow. The silica was placed into a flask and stirred in ammonium hydroxide (100 mL) and dichloromethane (100 mL) for 10 min. The silica was filtered and washed with additional ammonium hydroxide (50 mL) and dichloromethane (50 mL). The phases were separated and the aqueous layer was extracted an additional three times. The combined organic layers were washed twice with water, dried over Na₂SO₄, filtered and concentrated by rotary evaporation. Purification by column chromatography (25%, EtOAc:Hexanes) yielded 27 mg (0.18 mmol, 54%) of compound 4 as a mixture of diastereomers which was stored as a solution in MeOH at −15° C.

Although the invention is illustrated and described herein with reference to specific embodiments, the invention is not intended to be limited to the details shown. Rather, various modifications may be made in the details within the scope and range of equivalents of the claims and without departing from the invention.

1.-42. (canceled)

43. A compound having orae of the following structures: 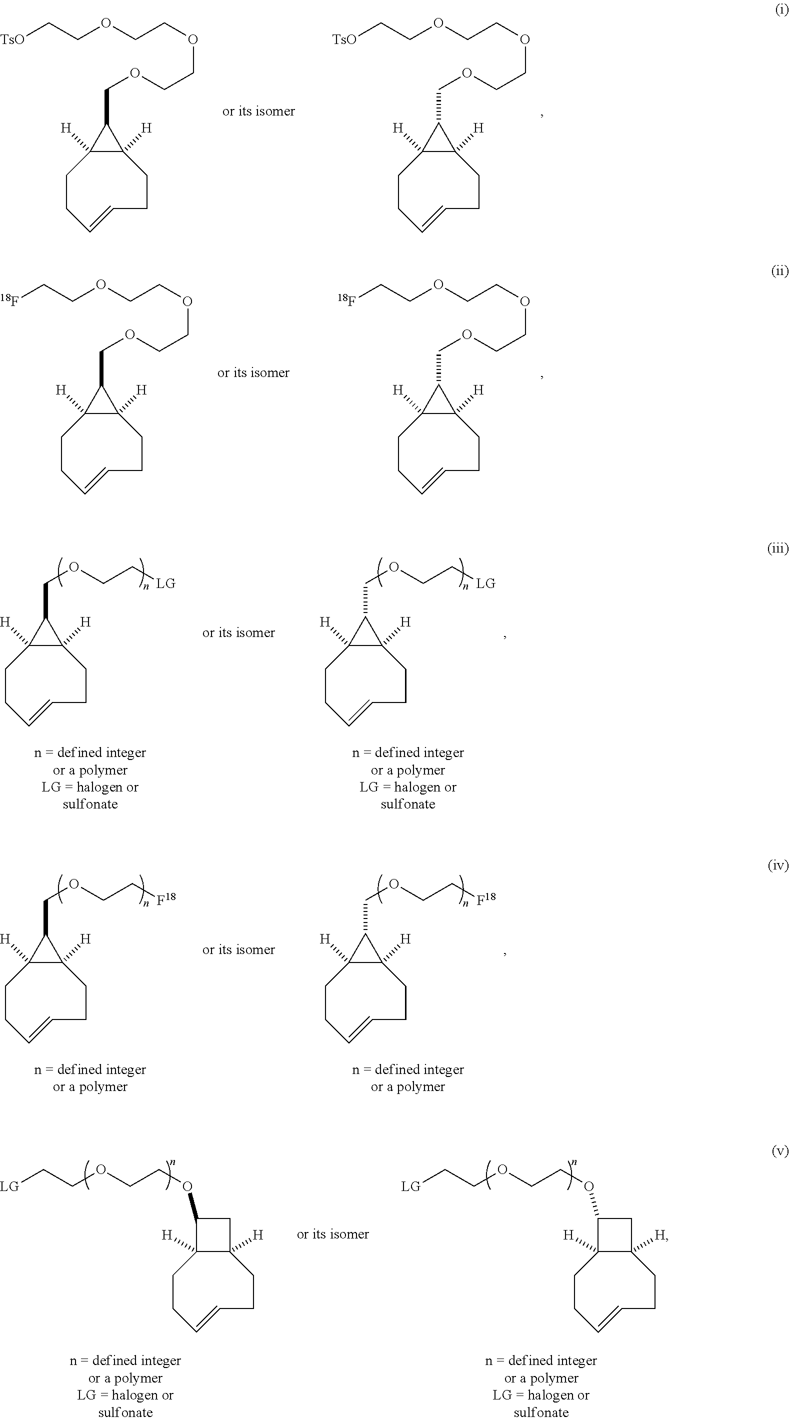 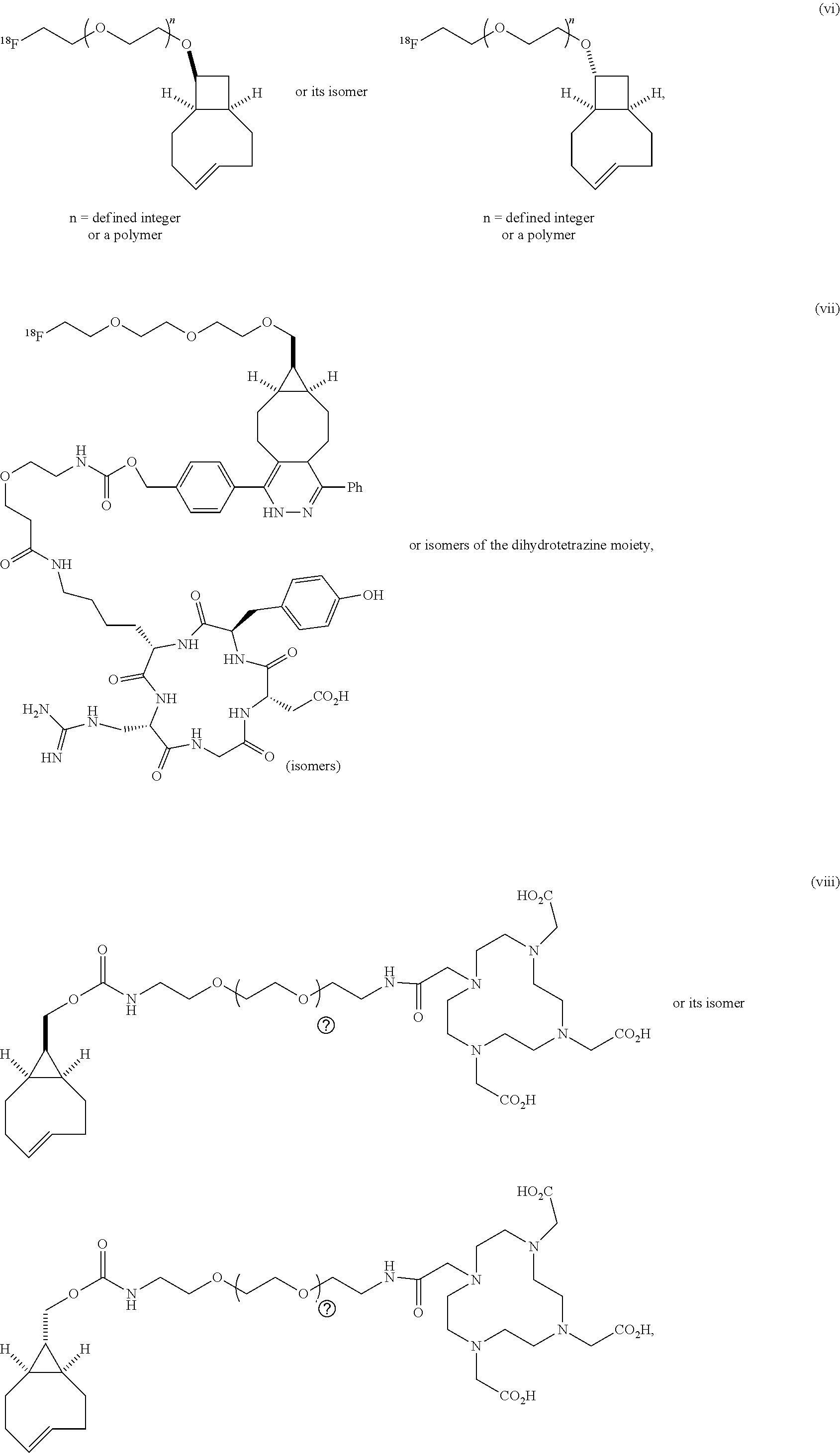 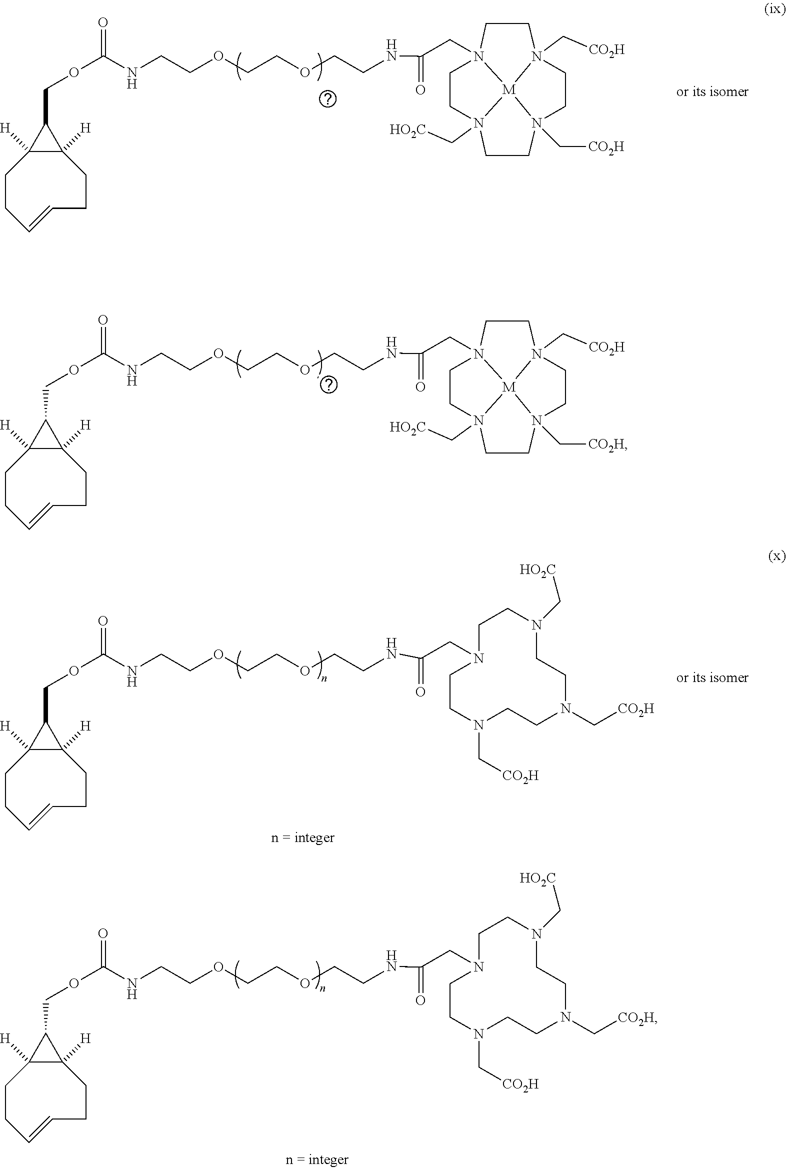 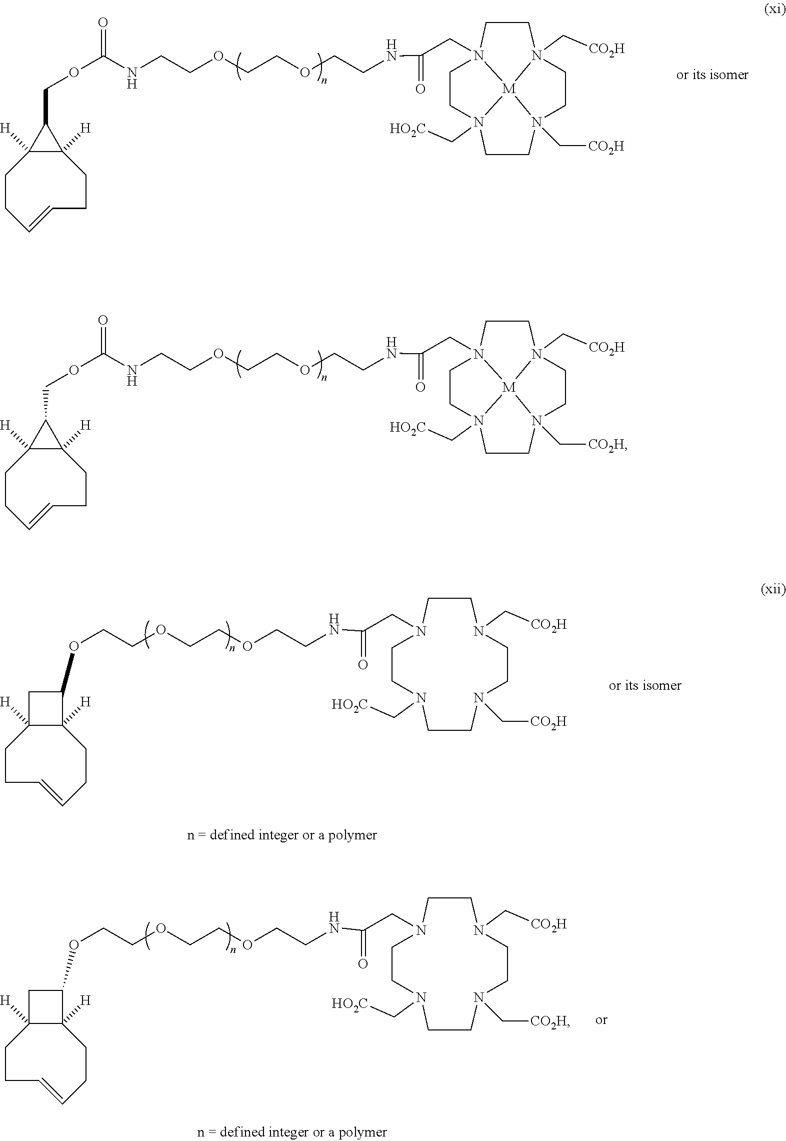 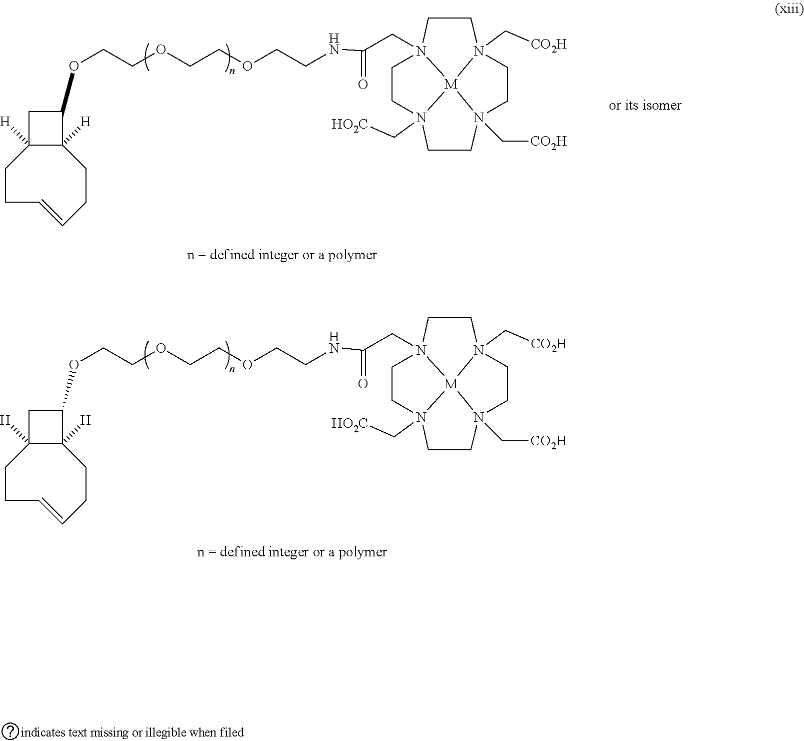
wherein M is a radioactive or a non-radioactive isotope of a metal.

44. The compound according to claim 43, wherein the metal comprises ⁶⁴Cu, ⁵⁷Cu, ⁸⁶Y, ⁹⁰Y, ¹⁷⁷Lu, Gd and Ln.

45. A compound having one sf the following structures: 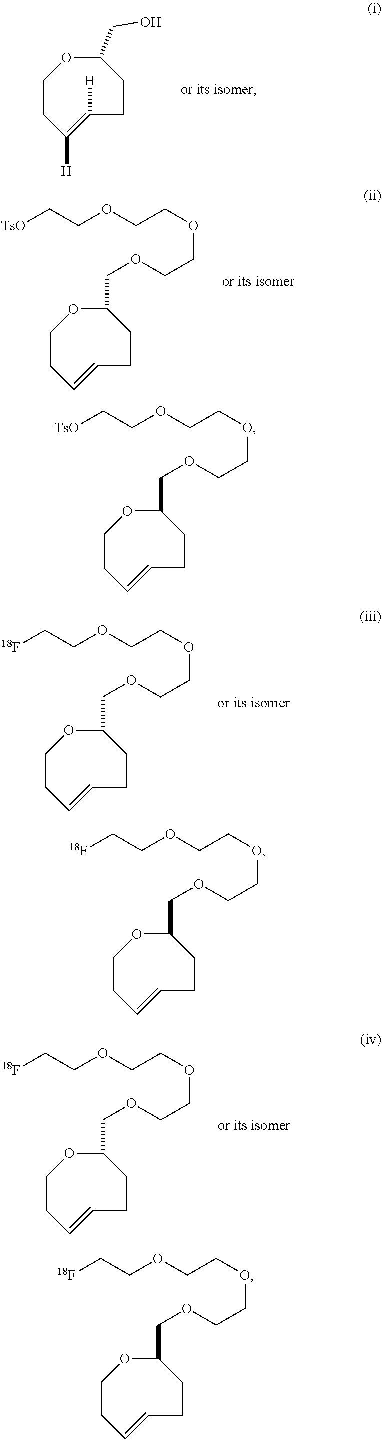 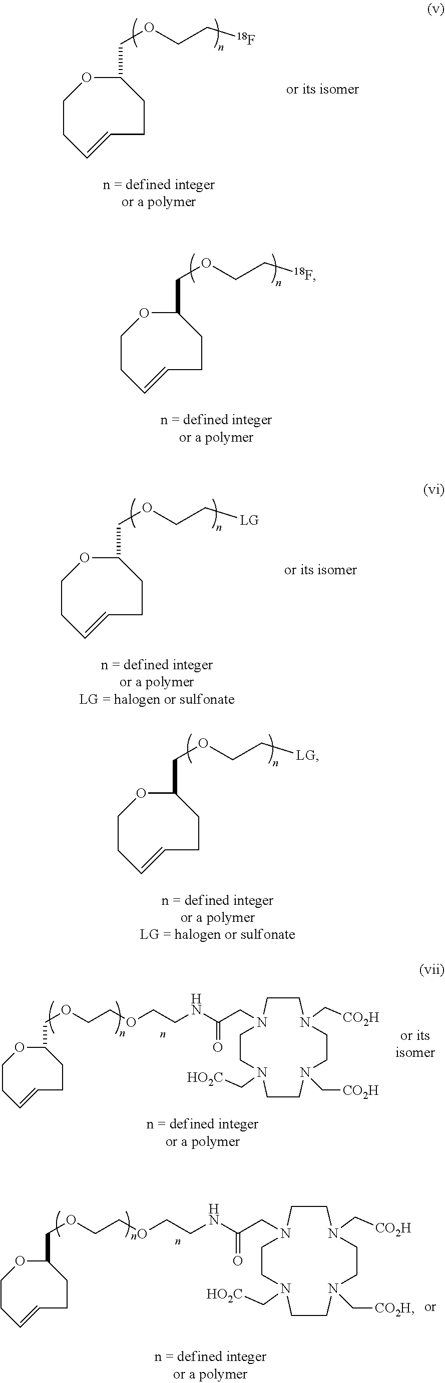 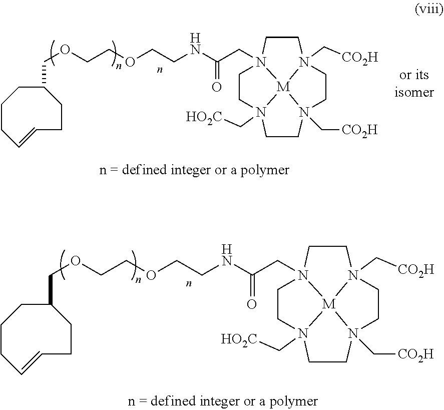
wherein M is a radioactive or a non-radioactive isotope of a metal.

46. The compound according to claim 45, wherein the metal comprises ⁶⁴Cu, ⁶⁷Cu, ⁸⁶Y, ⁹⁰Y, ¹⁷⁷Lu, Gd and Ln.

47. A compound having one of the following structures: 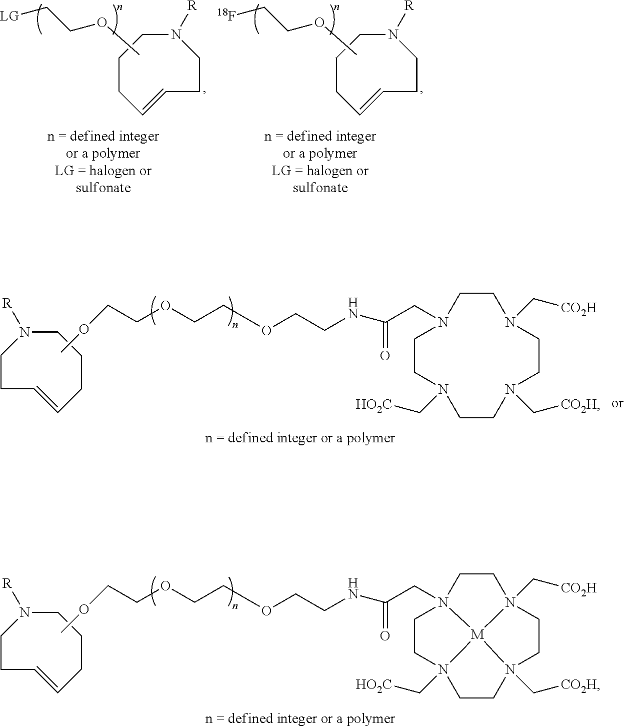
wherein M is a radioactive or a non-radioactive isotope of a metal, and
wherein R is chosen from hydrogen and methyl.

48. The compound according to claim 47, wherein the metal comprises ⁶⁴Cu, ⁶⁷Cu, ⁸⁶Y, ⁹⁰Y, ¹⁷⁷Lu, Gd and Ln.

49. A compound having one of the following structures: 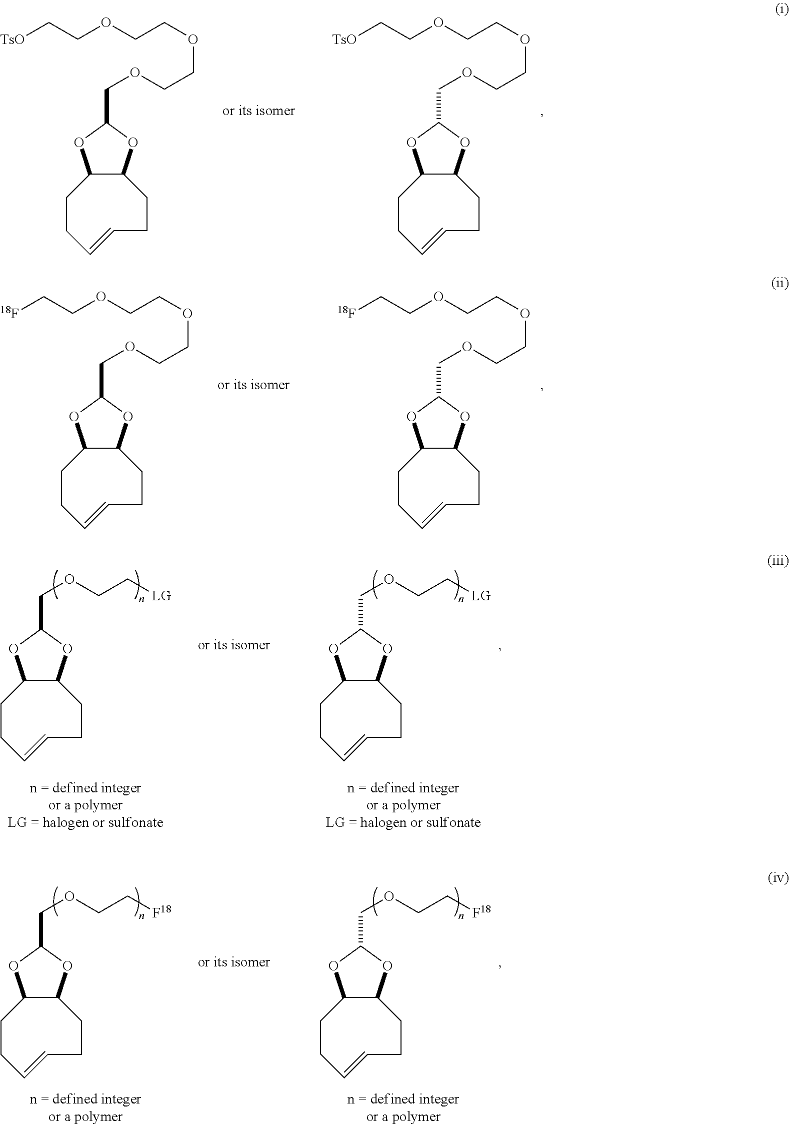 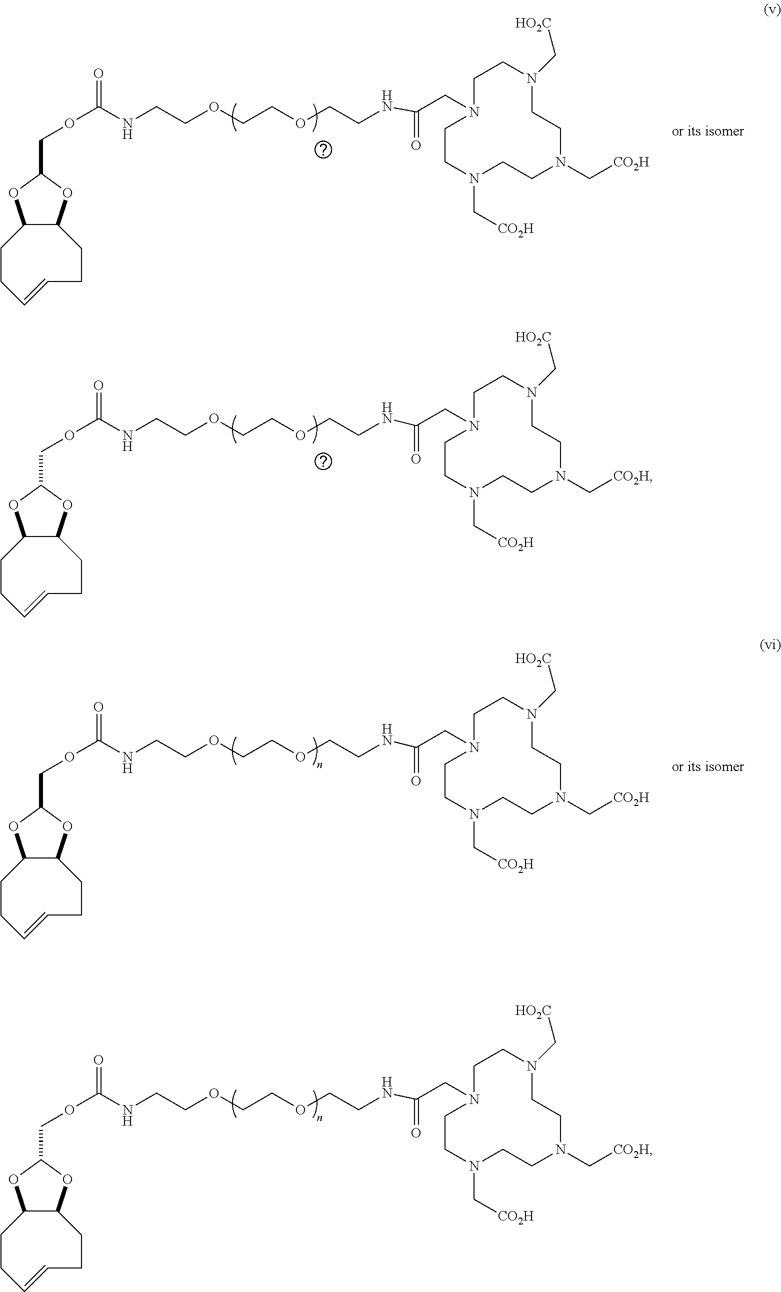 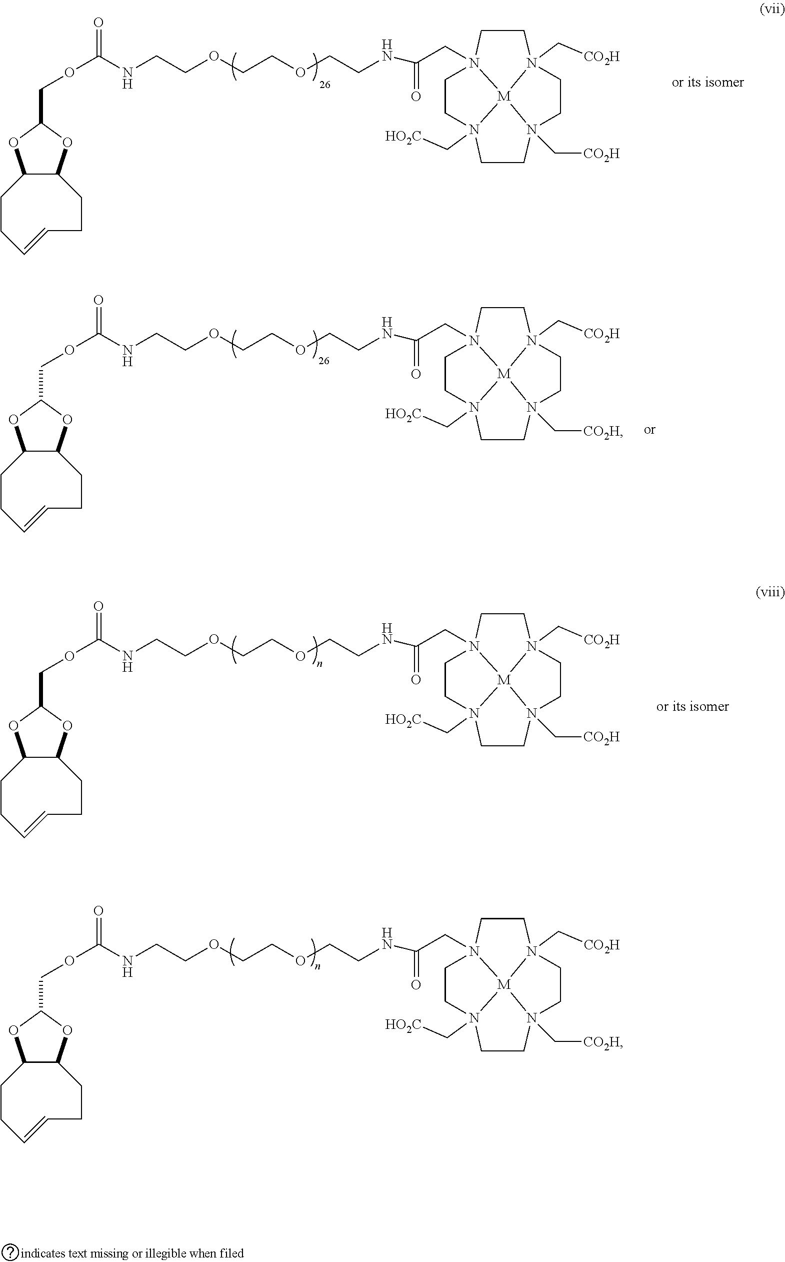
wherein M is a radioactive or a non-radioactive isotope of a metal.

50. The compound according to claim 49, wherein the metal comprises ⁶⁴Cu, ⁶⁷Cu, ⁸⁶Y, ⁹⁰, ¹⁷⁷Lu, Gd and Ln,

51. A compound having one of the following structures: 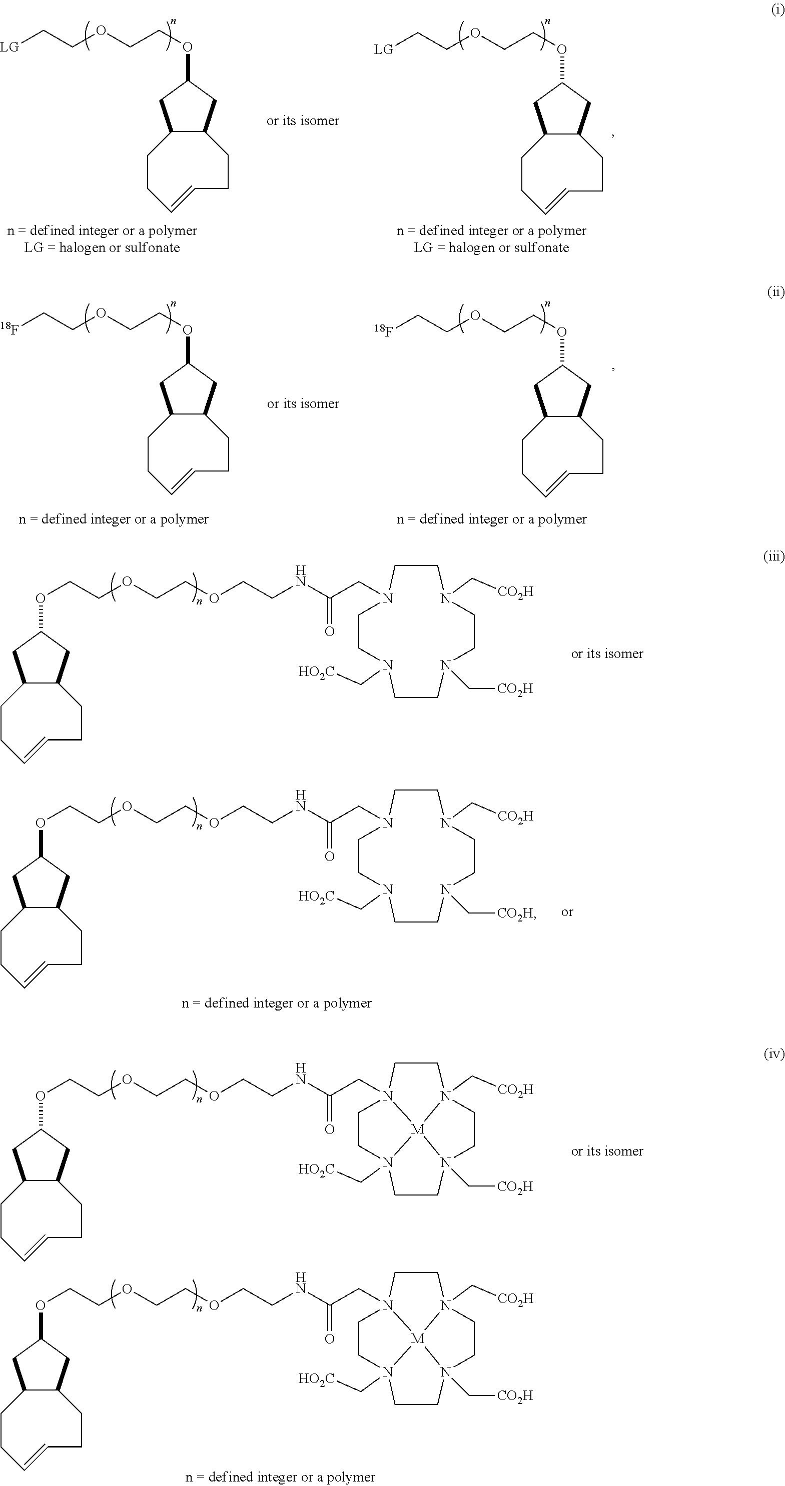
wherein M is a radioactive or a non-radioactive isotope of a metal.

52. The compound according to claim 43, wherein the metal comprises ⁶⁴Cu, ⁶⁷Cu, ⁸⁶Y, ⁹⁰Y, ¹⁷⁷Lu, Gd and Ln.

53. A compound having one of the following structures: 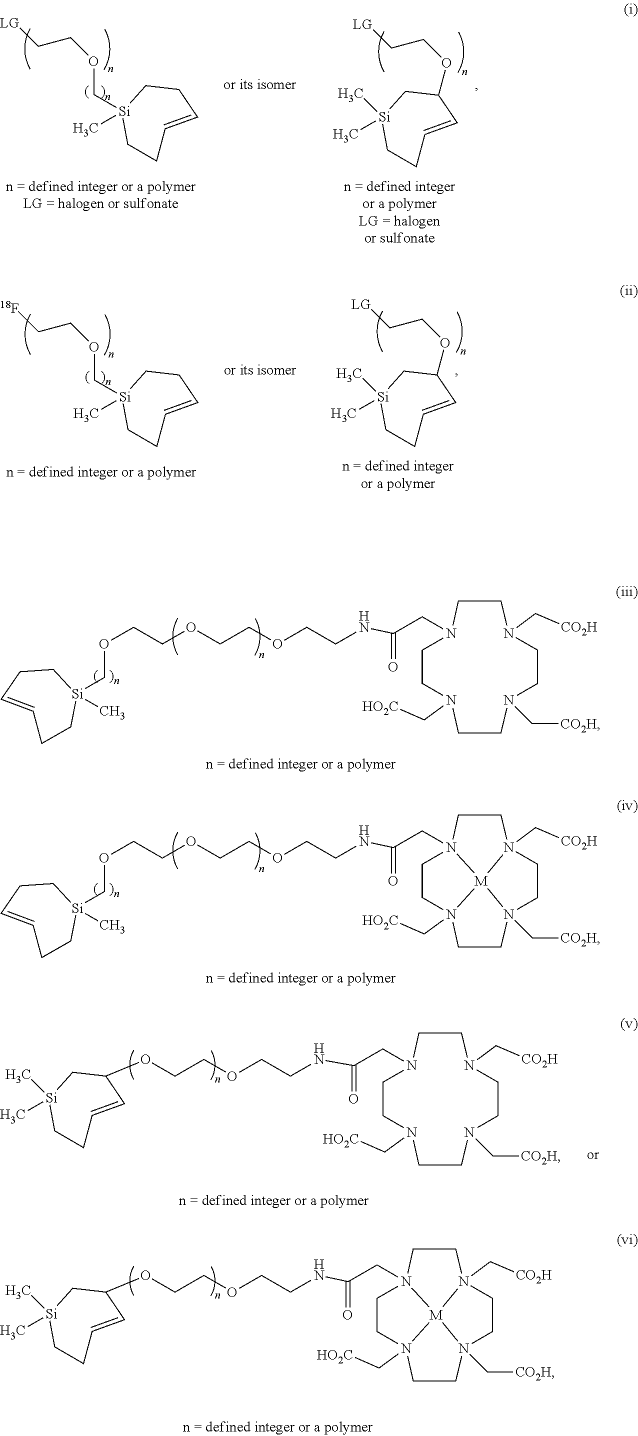
wherein M is a radioactive or a non-radioactive isotope of a metal.

54. The compound according to claim 43, wherein the metal comprises ⁶⁴Cu, ⁶⁷Cu, ⁸⁶Y, ⁹⁹Y, ¹⁷⁷Lu, Gd, and Ln.

55. A compound having one of the following structures: 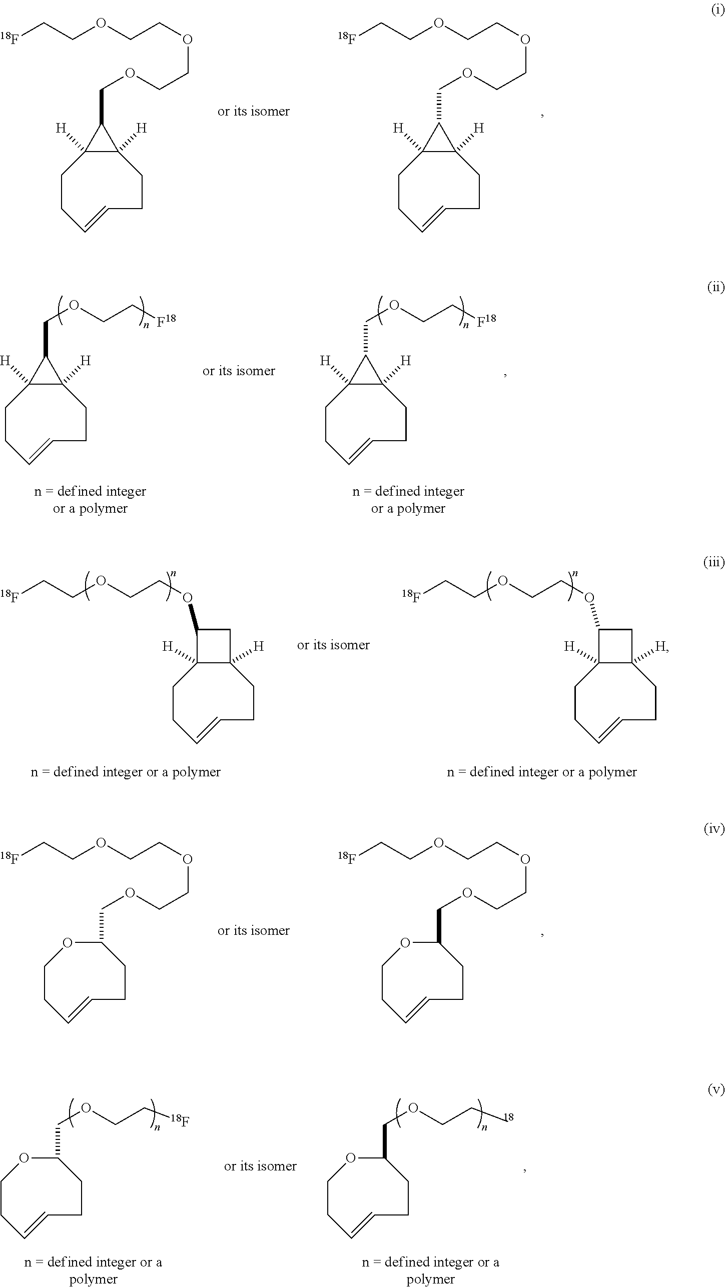 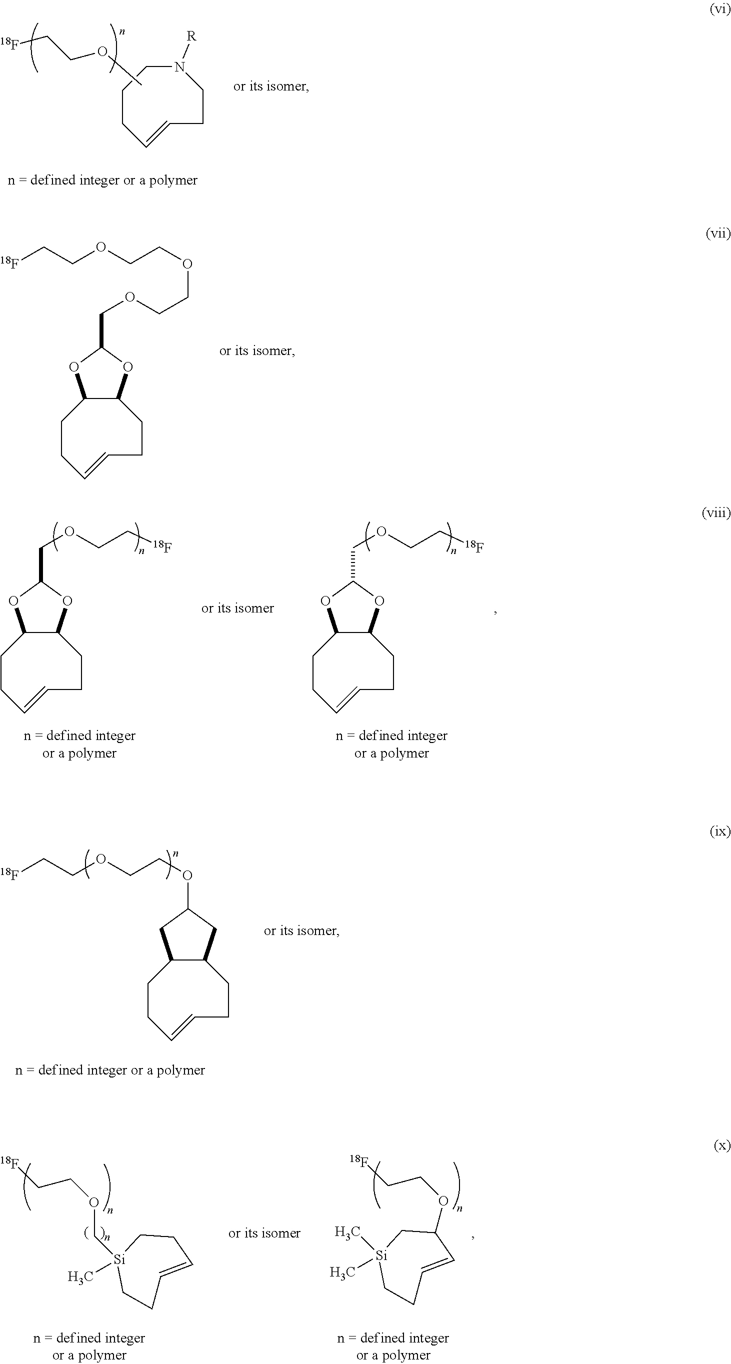 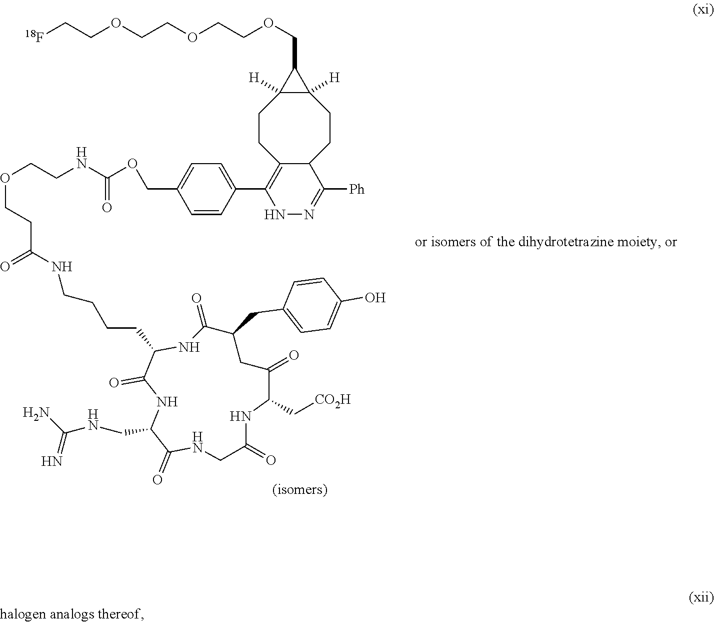 [SMILES: N=C(N)NC[C@@H]1NC(=O)[C@H](CCCCNC(=O)CCOCCNC(=O)OCc2ccc(C3=C4CC[C@@H]5[C@H](CCC4C(c4ccccc4)=NN3)[C@@H]5COCCOCCOCC[18F])cc2)NC(=O)[C@@H](Cc2ccc(O)cc2)CC(=O)[C@H](CC(=O)O)NC(=O)CNC1=O ; "or isomers of the dihydrotetrazine moiety, or"]
wherein the halogen is an isotope of chlorine, bromine, iodine or astatine, and
wherein R is chosen from hydrogen and methyl.

56. A Diels-Alder conjugate of the compound according to claim 55 and a tetrazine.

57. A radiotracer for PET imaging comprising the compound according to claim 55 and derivatives thereof.

58. A method of PET imaging, comprising injecting into a subject in need of said imaging an ¹⁸F compound having one of the following structures: 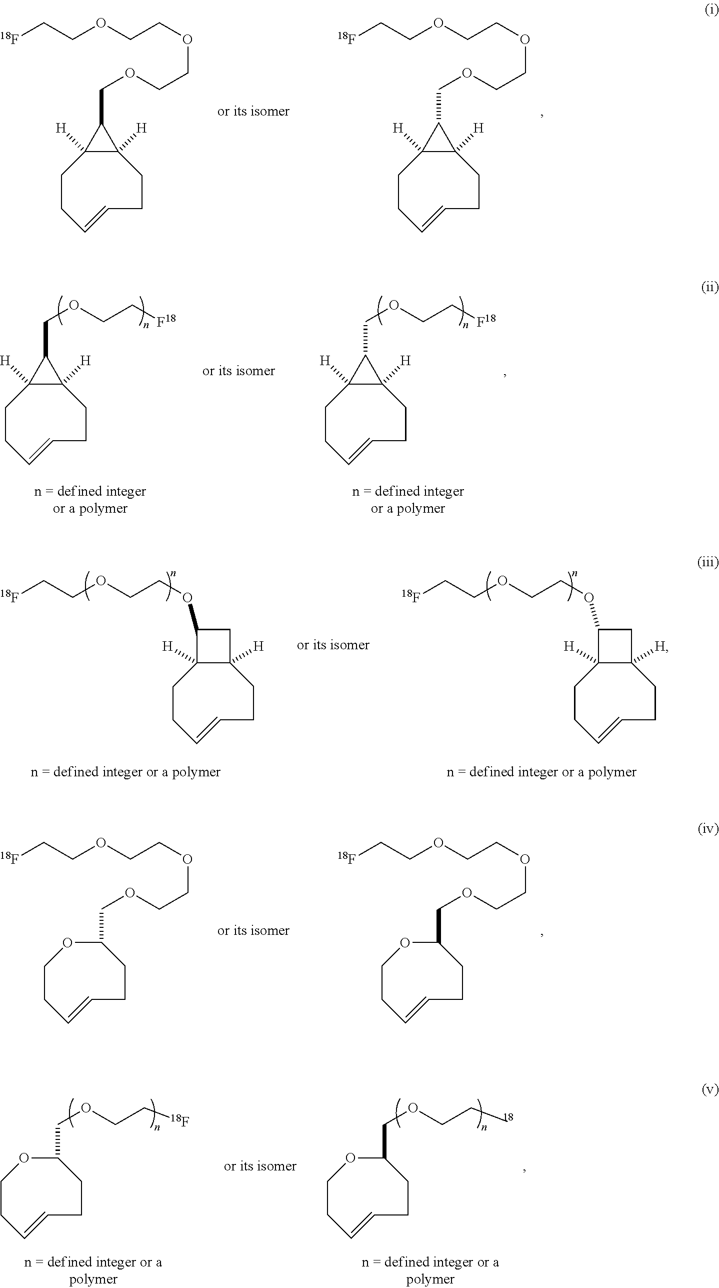 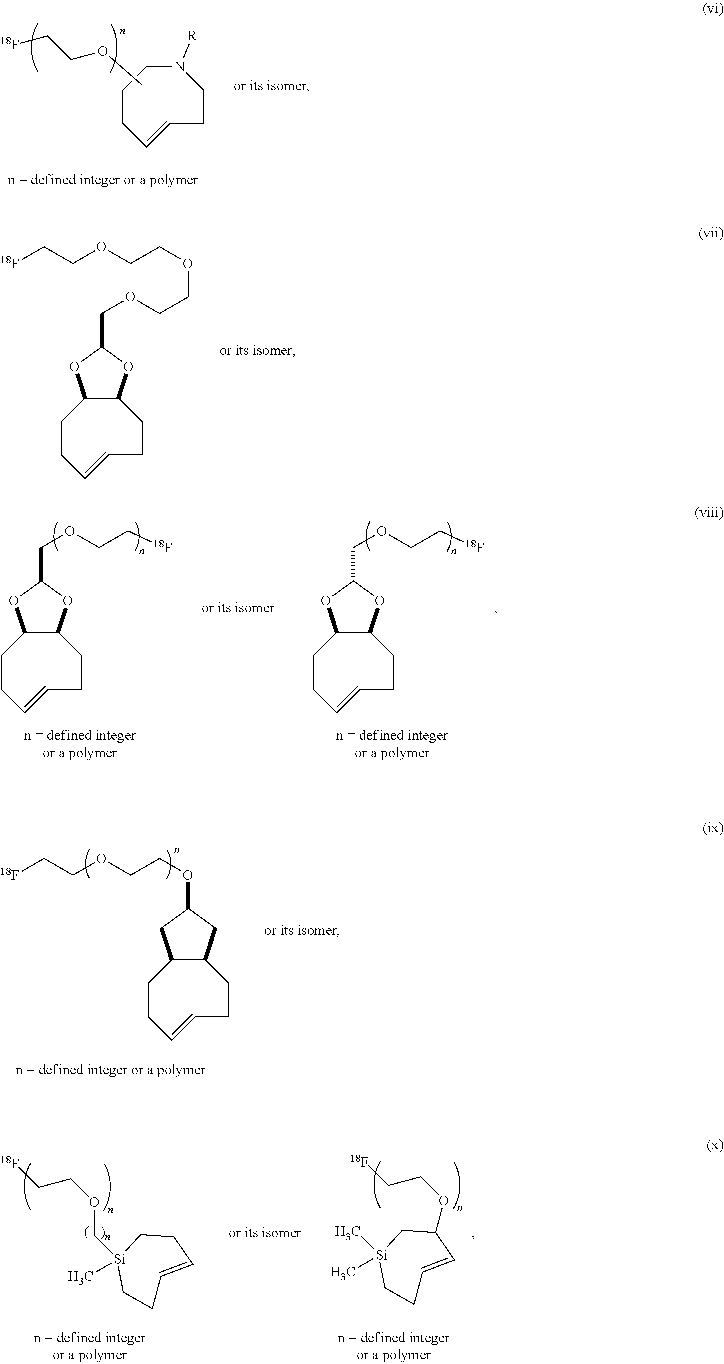 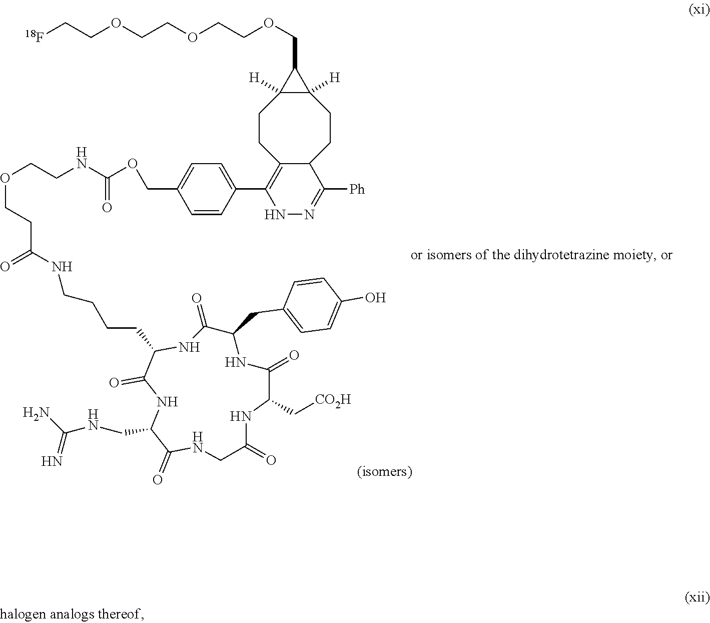 [SMILES: N=C(N)NC[C@@H]1NC(=O)[C@H](CCCCNC(=O)CCOCCNC(=O)OCc2ccc(C3=C4CC[C@@H]5[C@H](CCC4C(c4ccccc4)=NN3)[C@@H]5COCCOCCOCC[18F])cc2)NC(=O)[C@@H](Cc2ccc(O)cc2)NC(=O)[C@H](CC(=O)O)NC(=O)CNC1=O ; "or isomers of the dihydrotetrazine moiety, or"]
wherein the halogen is an isotope of chlorine, bromine, iodine or astatine, and
wherein R is chosen from hydrogen and methyl.
